(19) United States
(12) Patent Application Publication
(10) Pub. No.: US 2014/0120075 A1
(43) Pub. Date: May 1, 2014

(54) Nanozyme Compositions and Methods of Synthesis and Use Thereof

(71) Applicant: Board of Regents of the University of Nebraska, Lincoln, NE (US)

(72) Inventors: Alexander V. Kabanov, Chapel Hill, NC (US); Davika Soundara Manickam, Omaha, NE (US); Anna M. Brynskikh, Omaha, NE (US)

(73) Assignee: Board of Regents of the University of Nebraska, Lincoln, NE (US)

(21) Appl. No.: 14/119,368

(22) PCT Filed: May 24, 2012
(86) PCT No.: PCT/US2012/039325

§ 371 Date: Dec. 27, 2013

Related U.S. Application Data
(60) Provisional application No. 61/489,356, filed on May 24, 2011.

(51) Int. Cl. A61K 47/48

(52) U.S. Cl. CPC A61K 47/48315
USPC 424/94.3; 435/188

(57) ABSTRACT

Nanozymes and methods of use and synthesis thereof are provided.

This application claims priority under 35 U.S.C. §119(e) to U.S. Provisional Patent Application No. 61/489,356, filed on May 24, 2011. The foregoing application is incorporated by reference herein.

This invention was made with government support under Grant No. P20 RR021937-01A2 awarded by the National Institutes of Health. The government has certain rights in the invention.

### FIELD OF THE INVENTION

The present invention relates to the transport of biologically active proteins across biological membranes, particularly across the blood-brain barrier.

### BACKGROUND OF THE INVENTION

Attenuation of oxidative stress and inflammation is a promising strategy to prevent brain tissue damage and treat central nervous system (CNS)-related disorders, including Parkinson's disease (PD), Alzheimer's disease, amyotrophic lateral sclerosis, as well as traumatic brain injury, stroke and transient ischemic attacks (Barnham et al. (2004) Nat. Rev. Drug Discov., 3:205-214; Gilgun-Sherki et al. (2001) Neuropharmacol., 40:959-975; Chrissobolis et al. (2011) Front. Biosci., 16:1733-1745; Kaur et al. (2011) Int. J. Biol. Med. Res., 2:611-615; Pan et al. (2007) Neuroradiol., 49:93-102). Antioxidant enzymes like copper-zinc superoxide dismutase (Cu/Zn SOD, also known as SOD 1) and catalase are potent scavengers of ROS. However, their delivery to the brain represents a major challenge because of their proteolytic degradation, immunogenicity, short circulation half-life and poor permeability across the blood-brain barrier (BBB) (Banks, W. A. (2008) Biopolymers, 90:589-594). Accordingly, superior compositions and methods for the delivery of antioxidant enzymes such as SOD1 are desired.

### SUMMARY OF THE INVENTION

In accordance with one aspect of the instant invention, methods of synthesizing a nanozyme are provided. In a particular embodiment, the method comprises complexing at least one block copolymer and at least one protein of interest (e.g., an antioxidant enzyme such as SOD or catalase), linking the block copolymer with the protein of interest with a cross-linker, and purifying the generated nanozymes. In a particular embodiment, the block copolymer comprises at least one ionically charged (e.g., cationic) polymeric segment and at least one hydrophilic polymeric segment. The cationic polymeric segment may comprise cationic amino acids (e.g., poly-lysine). In a particular embodiment, the purification step is performed by size exclusion chromatography and/or centrifugal filtration.

In accordance with another aspect of the instant invention, isolated nanozymes synthesized by the instant methods are also provided. Compositions comprising the nanozymes of the instant invention are also provided.

In accordance with another aspect of the instant invention, methods of treating a reactive oxygen species (ROS)-related disease/disorder in a subject are provided. In a particular embodiment, the method comprises administering at least one nanozyme of the instant invention to a subject.

### BRIEF DESCRIPTIONS OF THE DRAWING

FIG. 1 provides a schematic representation of spontaneous formation of block ionomer complexes (BICs) through electrostatic binding of a negatively charged enzyme with a cationic block copolymer followed by covalent cross-linking to obtain cl-nanozymes. The scheme implies 1) each BIC contains one protein globule and 2) the particle size may further increase upon cross-linking.

FIG. 2 provides images of gel retardation analyses. SOD1 (5 μg/lane; FIG. 2A) and catalase (3 μg/lane; FIG. 2B) were loaded on a denaturing polyacrylamide gel and protein bands were stained using SYPRO® Ruby. nS or nC—native enzymes SOD1 or catalase; PEG-pLL₅₀—free block copolymer; S1 or C1—non-cross-linked BIC; S2 or C2 and S3 or C3—cl-nanozymes cross-linked using DTSSP and BS³, respectively. Selected samples as indicated were treated with DTT (25 mM) for 30 minutes prior to gel electrophoresis.

FIG. 3 provides an image of a gel retardation analysis. SOD1 (S) (5 μg/lane) and catalase (C) (3 μg/lane) were loaded on a denaturing polyacrylamide gel and protein bands were stained using SYPRO® Ruby. nS or nC—native enzyme SOD1 or catalase; PEG-pLL₅₀—free block copolymer; S1 or C1—non-cross-linked BIC; S2 or C2—DTSSP-cross-linked cl-nanozymes; subscript “p” refers to the respective purified forms.

FIG. 4 provides typical dose response plots showing inhibition of PG autoxidation by SOD1 (FIG. 4A) and H₂O₂ decomposition by catalase (FIG. 4B). ΔA240/minute indicates rate of the decomposition reaction. nS and nC—native SOD1 and catalase; S1 and C1—their non-cross-linked BICs; S2, C2 and S3, C3—DTSSP and BS³-cross-linked cl-nanozymes, respectively. Inhibition of PG autoxidation by added SOD1 was monitored at 420 nm whereas H₂O₂ decomposition by added catalase was monitored at 240 nm.

FIG. 5 provides FPLC chromatogram profiles depicting purification of DTSSP-cross-linked SOD1 cl-nanozyme (S2; FIG. 5A) and catalase cl-nanozyme (C2; FIG. 5B). S2 or C2 was loaded onto a HiPrep 16/60 Sephacryl™ S-400 HR column and eluted using 10 mM HBS (pH 7.4) at a flow rate of 0.5 mL/minute. AUC analysis determined the proportion of each fraction in the sample.

FIG. 6 provides representative DLS plots showing effects of purification on size distribution of DTSSP-cross-linked SOD1 cl-nanozymes (S2; FIG. 6A) and catalase cl-nanozymes (C2; FIG. 6B). Subscript “p” refers to the respective purified forms. Tables in the inset show D_{eff} and PDI measured at 0.1 mg/mL enzyme concentration in 10 mM HBS (pH 7.4).

FIG. 7 shows the morphology of DTSSP-cross-linked SOD1 cl-nanozyme (S2; FIG. 7A) and catalase cl-nanozyme (C2; FIG. 7B) observed under AFM. Subscript ‘p’ refers to the respective purified forms. Samples deposited on APS mica were scanned using a Multimode NanoScope IV system operated in tapping mode.

FIG. 8 provides sedimentation equilibrium analysis of DTSSP-cross-linked SOD1 cl-nanozymes before (FIG. 8A) and after (FIG. 8B) purification. The sample concentration in HBS at equilibrium is shown as a function of radius. The solid lines are theoretical curves and figure insets show molecular weight and speed at which equilibrium was attained.

FIG. 9 shows the cytotoxicity of SOD1 formulations determined in TBMEC monolayers (FIG. 9A) and CATH.a neurons (FIG. 9B). nS—native enzyme; PEG-pLL₅₀—free block copolymer; S1—non-cross-linked BIC; S2p and S3p—DTSSP- and BS³-cross-linked purified cl-nanozymes. Cells were treated for 24 hours as indicated following which cell viability was determined using a MTS assay kit.

FIG. 10 shows the cytotoxicity of SOD1 formulations in TBMEC monolayers. S2 and S2p designate DTSSP-cross-linked cl-nanozymes before and after purification, respectively. Cells were treated for 24 hours as indicated following which cell viability was determined using a MTS assay kit.

FIG. 11 shows the superoxide scavenging by SOD1 formulations in TBMEC monolayers (FIG. 11A) and CATH.a neurons (FIG. 11B). Cells were treated for 2 hours with native SOD1 or its formulations diluted in complete culture medium, washed and then incubated in fresh medium for different times. nS —native enzyme; S1—non-cross-linked BIC; S2p and S3p—DTSSP- and BS³-cross-linked-purified cl-nanozymes. O₂.⁻ levels are expressed as % relative to untreated cells. In cells treated with S2p and S3p (FIG. 11A) and S2p (FIG. 11B) the decreases in O₂.⁻ are statistically significant (P<0.05) compared to those treated with nS and S1, except for S3p at the 2 hour time point (TBMEC) and S2p at the 0 hour time point (Cath.a).

FIG. 12 shows the therapeutic efficacy in a rat MCAO model. FIG. 12A provides TTC staining of brain slices. FIG. 12B provides the quantitative representation of infarct size. FIG. 12C shows functional outcomes assessed using a sensorimotor score. nS—native SOD1; S2p—DTSSP-cross-linked purified cl-nanozymes. Treatment was administered at the onset of reperfusion (following a 2 hour ischemia) i.v. via the tail vein at a dose of 10 kU/kg and sensorimotor functions were evaluated 22 hours post-reperfusion before dissecting the brains for TTC staining.

### DETAILED DESCRIPTION OF THE INVENTION

Development of well-defined nanomedicines is critical for their successful clinical translation. A simple synthesis and purification procedure is established herein for chemically cross-linked polyion complexes of Cu/Zn superoxide dismutase (SOD1) or catalase with a cationic block copolymer, methoxy-poly(ethylene glycol)-block-poly(L-lysine hydrochloride) (PEG-pLL₅₀). Such complexes, termed cross-linked nanozymes (cl-nanozymes) retain catalytic activity and have narrow size distribution. Moreover, their cytotoxicity is decreased compared to non-cross-linked complexes due to suppression of release of the free block copolymer. SOD1 cl-nanozymes exhibit prolonged ability to scavenge experimentally induced reactive oxygen species (ROS) in cultured brain microvessel endothelial cells and central neurons. In vivo they decrease ischemia/reperfusion-induced tissue injury and improve sensorimotor functions in a rat middle cerebral artery occlusion (MCAO) model after a single intravenous (i.v.) injection. Altogether, well-defined cl-nanozymes are modalities for attenuation of oxidative stress after brain injury.

Even though the BBB can be partially compromised in stroke, it still remains the key impediment for CNS transport of enzymes (Sood et al. (2009) J. Cereb. Blood Flow Metab., 29:308-316; Zhang et al. (2001) Brain Res., 889:49-56). Several strategies have been explored to improve delivery of antioxidant enzymes including PEGylation (Beckman et al. (1988) J. Biol. Chem., 263:6884-6892; Veronese et al. (2002) Adv. Drug Deliv. Rev., 54:587-606), use of fusion constructs with protein transduction domains (Eum et al. (2004) Free Radic. Biol. Med., 37:1656-1669; Grey et al. (2009) FEBS J., 276:6195-6203; Kim et al. (2009) Free Radic. Biol. Med., 47:941-952; Lu et al. (2006) J. Biol. Chem., 281:13620-13627), encapsulation in liposomes (Corvo et al. (1999) Biochim. Biophys. Acta, 1419:325-334; Freeman et al. (1983) J. Biol. Chem., 258:12534-12542; Imaizumi et al. (1990) Stroke 21:1312-1317), or poly(D,L-lactide-co-glycolide) (PLGA) nanoparticles (Reddy et al. (2009) FASEB J., 23:1384-1395), lecithinization (Igarashi et al. (1994) J. Pharmacol. Exp. Ther., 271:1672-1677; Ishihara et al. (2009) J. Pharmacol. Exp. Ther., 328:152-164; Koo et al. (2001) Kidney Int., 60:786-796), or conjugation with antibodies (immunotargeting). PEP-1-SOD1 and PEP-1-catalase fusion constructs attenuated ischemic neuronal damage in vivo (Eum et al. (2004) Free Radic. Biol. Med., 37:1656-1669; Kim et al. (2009) Free Radic. Biol. Med., 47:941-952). However, such constructs have relatively low stability in circulation and can induce immune responses in patients (Reddy et al. (2009) FASEB J., 23:1384-1395). The circulation time and stability of proteins can be increased to several hours by PEGylation—modification of the protein with poly(ethylene glycol) (PEG). However, such modification also drastically decreases permeability of proteins across the brain microvessels and entry into brain cells, thereby hindering therapeutic effects of PEGylated SOD1 after cerebral ischemia/reperfusion injury (Veronese et al. (2002) Adv. Drug Deliv. Rev., 54:587-606; Francis et al. (1997) Exp. Neurol., 146:435-443).

The constitutive vascular expression of platelet-endothelial adhesion molecule (PE-CAM)-1 and intercellular adhesion molecule (ICAM)-1 has been used for catalase delivery to the endothelium using multivalent conjugates of catalase with anti-PECAM or anti-ICAM antibodies (Atochina et al. (1998) Am. J. Physiol., 275:L806-817; Christofidou-Solomidou et al. (2003) Am. J. Physiol. Lung Cell Mol. Physiol., 285:L283-292; Muzykantov et al. (1996) Proc. Natl. Acad. Sci., 93:5213-5218; Shuvaev et al. (2004) Methods Mol. Biol., 283:3-19) or coating catalase-loaded nanoparticles with these antibodies (Moro et al. (2003) Am. J. Physiol. Cell. Physiol., 285:C1339-1347). These conjugates or nanoparticles were internalized by endothelial cells, remained functionally active and protected pulmonary vasculature against acute oxidative stress (Christofidou-Solomidou et al. (2003) Am. J. Physiol. Lung Cell Mol. Physiol., 285:L283-292; Muzykantov et al. (1996) Proc. Natl. Acad. Sci., 93:5213-5218). More recently, it has been reported that SOD1 and catalase immobilized in magnetic nanoparticles were stable against proteolytic degradation, transported into endothelial cells in vitro and rescued these cells from H₂O₂-induced oxidative stress (Chorny et al. (2010) J. Control Release, 146:144-151).

In another approach SOD1 incorporated into PLGA nanoparticles was shown to reduce ischemic brain injury after intracarotid (i.c.) injection (Reddy et al. (2009) FASEB J., 23:1384-1395). However, PLGA-matrix hinders access of the substrate to the enzyme active sites. Furthermore, instability of proteins in such formulations, especially upon PLGA hydrolysis may limit their utility (Jiang et al. (2008) Mol. Pharm., 5:808-817). Finally, cationic liposome-entrapped SOD1 was shown to reduce infarction upon cerebral ischemia in rats, but low stability of this formulation and possible toxicity impeded its further use (Reddy et al. (2009) FASEB J., 23:1384-1395; Sinha et al. (2001) Biomed. Pharmacother., 55:264-271).

A distinct class of catalytic nanoparticles has been developed based on polyion complexes (also known as “block ionomer complexes”, BICs) of enzymes and cationic block copolymers (nanozymes) and their potential to treat PD (using catalase nanozymes loaded in cell carriers), Angiotensin-II hypertension (using SOD 1 nanozymes), and delivery of active butyrylcholinesterase enzyme to the brain in healthy mice has been demonstrated (Batrakova et al. (2007) Bioconjug. Chem., 18:1498-1506; Rosenbaugh et al. (2010) Biomaterials 31:5218-5226; Gaydess et al. (2010) Chem. Biol. Interact., 187:295-298; Haney et al. (2011) Nanomed., 6:1215-1230; Zhao et al. (2011) Nanomed., 6:25-42). More recently, it has been reported that covalently stabilized cl-nanozymes of SOD1 improved stability (in the blood and brain tissue) and delivered SOD1 to the brain parenchyma in healthy mice (Klyachko et al. (2012) Nanomed. Nanotechnol. Biol. Med., 8:119-129).

It is shown herein that the purification of cl-nanozymes through the removal of non-cross-linked species results in a homogenous sample with well-defined chemical composition and physicochemical characteristics, which further increases its stability against dissociation, improves its in vivo disposition and enhances overall efficacy of the delivery process. In other words, the purification method leads to the production of “pharmaceutical-grade” entities. Herein, well-defined antioxidant cl-nanozymes containing SOD1 or catalase were synthesized and characterized. In particular, the behavior of selected formulations in an in vitro model of cultured brain microvessel endothelial cells and central neurons was studied. Further, the therapeutic efficacy of SOD1 cl-nanozymes was demonstrated in vivo in a rat MCAO model of ischemia/reperfusion injury.

In accordance with the present invention, compositions and methods are provided for the transport of biologically active proteins (e.g., SOD or catalase) across biological membranes, particularly across the blood-brain barrier. Methods are also provided for the administration of nanozymes of the instant invention comprising a therapeutic protein to a patient in order to treat conditions in which the therapeutic protein is known to be effective. In a particular embodiment, the nanozymes are administered systemically.

### I. DEFINITIONS

The following definitions are provided to facilitate an understanding of the present invention:

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition resulting in a decrease in the probability that the subject will develop the condition.

As used herein, the term “subject” refers to an animal, particularly a mammal, particularly a human.

A “therapeutically effective amount” of a compound or a pharmaceutical composition refers to an amount effective to prevent, inhibit, treat, or lessen the symptoms of a particular disorder or disease. The treatment of cancer herein may refer to curing, relieving, and/or preventing the cancer, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

As used herein, the term “amphiphilic” means the ability to dissolve in both water and lipids/apolar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). As used herein, the term “hydrophilic” means the ability to dissolve in water.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), bulking substance (e.g., lactose, mannitol), excipient, auxilliary agent or vehicle with which an active agent of the present invention is administered. Pharmaceutically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “purified” or “to purify” refers to the removal of contaminants or undesired compounds from a sample or composition. For example, purification can result in the removal of from about 70 to 90%, up to 100%, of the contaminants or undesired compounds from a sample or composition. In certain embodiments, at least 90%, 93%, 95%, 96%, 97%, 98%, 99%, 99.5%, or more of undesired compounds from a sample or composition are removed from a preparation.

As used herein, the term “traumatic brain injury” includes any trauma, e.g., post-head trauma, impact trauma, and other traumas, to the head such as, for example, traumas caused by accidents and/or sports injuries, concussive injuries, penetrating head wounds, etc.

As used herein, the term “antioxidant” refers to compounds that neutralize the activity of reactive oxygen species or inhibit the cellular damage done by the reactive species or their reactive byproducts or metabolites. The term “antioxidant” may also refer to compounds that inhibit, prevent, reduce or ameliorate oxidative reactions. Examples of antioxidants include, without limitation, antioxidant enzymes (e.g., SOD or catalase), vitamin E, vitamin C, ascorbyl palmitate, vitamin A, carotenoids, beta carotene, retinoids, xanthophylls, lutein, zeaxanthin, flavones, isoflavones, flavanones, flavonols, catechins, ginkgolides, anthocyanidins, proanthocyanidins, carnosol, carnosic acid, organosulfur compounds, allylcysteine, alliin, allicin, lipoic acid, omega-3 fatty acids, eicosapentaeneoic acid (EPA), docosahexaeneoic acid (DHA), tryptophan, arginine, isothiocyanates, quinones, ubiquinols, butylated hydroxytoluene (BHT), butylated hydroxyanisole (BHA), super-oxide dismutase mimetic (SODm), and coenzymes-Q.

The terms “reactive oxygen species,” or “oxidative species,” as used herein, refer to oxygen derivatives from oxygen metabolism or the transfer of electrons, resulting in the formation of “free radicals” (e.g., superoxide anion or hydroxyl radicals).

### II. NANOZYMES

The nanozymes of the instant invention comprise at least one block copolymer and at least one protein or compound. The block copolymer comprises at least one ionically charged polymeric segment and at least one non-ionically charged polymeric segment (e.g., hydrophilic segment). In a particular embodiment, the block copolymer has the structure A-B or B-A. The block copolymer may also comprise more than 2 blocks. For example, the block copolymer may have the structure A-B-A, wherein B is an ionically charged polymeric segment. In a particular embodiment, the segments of the block copolymer comprise about 10 to about 500 repeating units, about 20 to about 300 repeating units, about 20 to about 250 repeating units, about 20 to about 200 repeating units, or about 20 to about 100 repeating units.

The ionically charged polymeric segment may be cationic or anionic. The ionically charged polymeric segment may be selected from, without limitation, polymethylacrylic acid and its salts, polyacrylic acid and its salts, copolymers of acrylic acid and its salts, poly(phosphate), polyamino acids (e.g., polyglutamic acid, polyaspartic acid), polymalic acid, polylactic acid, homopolymers or copolymers or salts thereof of aspartic acid, 1,4-phenylenediacrylic acid, ciraconic acid, citraconic anhydride, trans-cinnamic acid, 4-hydroxy-3-methoxy cinnamic acid, p-hydroxy cinnamic acid, trans glutaconic acid, glutamic acid, itaconic acid, linoleic acid, linlenic acid, methacrylic acid, maleic acid, trans-β-hydromuconic acid, trans-trans muconic acid, oleic acid, vinylsulfonic acid, vinyl phosphonic acid, vinyl benzoic acid, and vinyl glycolic acid and the like and carboxylated dextran, sulfonated dextran, heparin and the like. Examples of polycationic segments include but are not limited to polymers and copolymers and their salts comprising units deriving from one or several monomers including, without limitation: primary, secondary and tertiary amines, each of which can be partially or completely quaternized forming quaternary ammonium salts. Examples of these monomers include, without limitation, cationic amino acids (e.g., lysine, arginine, histidine), alkyleneimines (e.g., ethyleneimine, propyleneimine, butileneimine, pentyleneimine, hexyleneimine, and the like), spermine, vinyl monomers (e.g., vinylcaprolactam, vinylpyridine, and the like), acrylates and methacrylates (e.g., N,N-dimethylaminoethyl acrylate, N,N-dimethylaminoethyl methacrylate, N,N-diethylaminoethyl acrylate, N,N-diethylaminoethyl methacrylate, t-butylaminoethyl methacrylate, acryloxyethyltrimethyl ammonium halide, acryloxyethyl-dimethylbenzyl ammonium halide, methacrylamidopropyltrimethyl ammonium halide and the like), allyl monomers (e.g. dimethyl diallyl ammonium chloride), aliphatic, heterocyclic or aromatic ionenes. In a particular embodiment, the ionically charged polymeric segment is cationic. In a particular embodiment, the cationic polymeric segment comprises cationic amino acids (e.g., poly-lysine).

Examples of non-ionically charged water soluble polymeric segments include, without limitation, polyetherglycols, poly(ethylene oxide), copolymers of ethylene oxide and propylene oxide, polysaccharides, polyvinyl alcohol, polyvinyl pyrrolidone, polyvinyltriazole, N-oxide of polyvinylpyridine, N-(2-hydroxypropyl)methacrylamide (HPMA), polyortho esters, polyglycerols, polyacrylamide, polyoxazolines, polyacroylmorpholine, and copolymers or derivatives thereof.

The nanozymes of the instant invention may be synthesized by 1) contacting at least one block copolymer with at least one protein, 2) contacting the complex formed between the block copolymer and protein with a cross-linker, and 3) purifying the generated nanozymes from the non cross-linked components. The term “cross-linker” refers to a molecule capable of forming a covalent linkage between compounds (e.g., polymer and protein). In a particular embodiment, the cross-linker forms covalent linkages (e.g., an amide bond) between amino groups of the ionically charged polymeric segment and carboxylic groups of the protein. In a particular embodiment, the cross-linker forms covalent linkages between amino groups of the ionically charged polymeric segment and amino groups of the protein. Cross-linkers are well known in the art. In a particular embodiment, the cross-linker is a titrimetric cross-linking reagent. The cross-linker may be a bifunctional, trifunctional, or multifunctional cross-linking reagent. Examples of cross-linkers are provided in U.S. Pat. No. 7,332,527. The cross-linker may be cleavable or biodegradable or it may be non-biodegradable or uncleavable under physiological conditions. In a particular embodiment, the cross-linker comprises a bond which may be cleaved in response to chemical stimuli (e.g., a disulfide bond that is degraded in the presence of intracellular glutathione). The cross-linkers may also be sensitive to pH (e.g., low pH). In a particular embodiment, the cross-linker is selected from the group consisting of linkers 3,3′-dithiobis(sulfosuccinimidylpropionate) (DTSSP) and bis(sulfosuccinimidyl)suberate (BS³). 1-Ethyl-3[3-dimethylaminopropyl]carbodiimide hydrochloride (EDC) and (N-hydroxysulfosuccinimide) may also be used for cross-linking reactions.

In order to minimize any undesired cross-linking with the amino groups of the protein, excess ionic (cationic) polymer can be used in the cross-linking reaction. In a particular embodiment, the molar ratio of crosslinker to the ionically charged polymeric segment is less than about 1.0, less than about 0.8, or less than about 0.5. In a particular embodiment, the molar ration is about 0.5.

After synthesis, the nanozymes of the instant invention are purified from non cross-linked components. The nanozymes may be purified by methods known in the art. For example, the nanozymes may be purified by size exclusion chromatography (e.g., using a Sephacryl™ S-400 column or equivalent thereof) and/or centrifugal filtration (e.g., using a 100 kDa or 1000 kDa molecular weight cutoff). In a particular embodiment, the nanozymes are purified such that at least 95%, 96%, 97%, 98%, 99%, 99.5%, or more of undesired components are removed from the sample. In a particular embodiment, the nanozymes are purified such that the polydispersity index (PDI) of the preparation is less than about 0.1, less than about 0.8, or less than about 0.05. In a particular embodiment, the purified nanozymes have a diameter of less than about 100 nm.

The instant invention encompasses compositions comprising at least one nanozyme of the instant invention (e.g., a purified nanozyme) and at least one pharmaceutically acceptable carrier. The compositions of the instant invention may further comprise other therapeutic agents.

The present invention also encompasses methods for preventing, inhibiting, and/or treating a disease or disorder in a subject. The pharmaceutical compositions of the instant invention can be administered to an animal, in particular a mammal, more particularly a human, in order to treat/inhibit/prevent the disease or disorder. The pharmaceutical compositions of the instant invention may also comprise at least one other bioactive agent, particularly at least one other therapeutic agent (e.g., antioxidant). The additional agent may also be administered in separate composition from the nanozymes of the instant invention. The compositions may be administered at the same time or at different times (e.g., sequentially).

While the instant invention generally describes the use of proteins in the nanozymes, it is also within the scope of the instant invention to use other therapeutic agents or compounds of interest in the nanozymes. The compound(s) can be, without limitation, a biological agent, detectable agents (e.g., imaging agents or contrast agents), or therapeutic agent. Such agents or compounds include, without limitation, polypeptides, peptides, glycoproteins, nucleic acids (DNA, RNA, oligonucleotides, plasmids, siRNA, etc.), synthetic and natural drugs, polysaccharides, small molecules, lipids, and the like. In a particular embodiment, the protein or compound has an opposite charge (e.g., overall charge) opposite to the ionically charged polymeric segment.

In a particular embodiment of the instant invention, the proteins of the nanozymes are therapeutic proteins, i.e., they effect amelioration and/or cure of a disease, disorder, pathology, and/or the symptoms associated therewith. In a particular embodiment, the protein is an antioxidant and/or a scavenger of reactive oxygen species (ROS). The proteins may have therapeutic value against, without limitation, neurological degenerative disorders, stroke (e.g., transient focal ischemic stroke), Alzheimer's disease, Parkinson's disease, Huntington's disease, trauma, infections, meningitis, encephalitis, gliomas, cancers (including brain metastasis), HIV, HIV associated dementia, HIV associated neurocognitive disorders, paralysis, amyotrophic lateral sclerosis, cardiovascular disease (including CNS-associated cardiovascular disease, hypertension, heart failure), prion disease, obesity, metabolic disorders, inflammatory disease, lung inflammation (e.g. that associated with influenza infection), and lysosomal diseases (such as, without limitation, Pompe disease, Niemann-Pick, Hunter syndrome (MPS II), Mucopolysaccharidosis I (MPS I), GM2-gangliosidoses, Gaucher disease, Sanfilippo syndrome (MPS IIIA), and Fabry disease). Examples of specific proteins include, without limitation, superoxide dismutase (SOD) or catalase (e.g., of mammalian, particularly human, origin), cytokines, leptin (Zhang et al. (1994) Nature, 372:425-432; Ahima et al. (1996) Nature, 382:250-252; Friedman and Halaas (1998) Nature, 395:763-770), enkephalin, growth factors (e.g., epidermal growth factor (EGF; Ferrari et al. (1990) Adv Exp Med Biol. 265:93-99), basic fibroblast growth factor (bFGF; Ferrari et al. (1991) J Neurosci Res. 30:493-497), nerve growth factor (NGF; Koliatsos et al. (1991) Ann Neurol. 30:831-840)), amyloid beta binders (e.g. antibodies), modulators of α-, β-, and/or γ-secretases, glial-derived neutrotrophic factor (GDNF; Schapira, A. H. (2003) Neurology 61:S56-63), vasoactive intestinal peptide (Dogrukol-Ak et al. (2003) Peptides 24:437-444), acid alpha-glucosidase (GAA; Amalfitano et al. (2001) Genet Med. 3:132-138), acid sphingomyelinase (Simonaro et al. (2002) Am J Hum Genet. 71:1413-1419), iduronate-2-sultatase (I2S; Muenzer et al. (2002) Acta Paediatr Suppl. 91:98-99), α-L-iduronidase (IDU; Wraith et al. (2004) J Pediatr. 144:581-588), 3-hexosaminidase A (HexA; Wicklow et al. (2004) Am J Med Genet. 127A:158-166), acid β-glucocerebrosidase (Grabowski, G. A., (2004) J Pediatr. 144:S15-19), N-acetylgalactosamine-4-sulfatase (Auclair et al. (2003) Mol Genet Metab. 78:163-174), and α-galactosidase A (Przybylska et al. (2004) J Gene Med. 6:85-92).

In a particular embodiment, methods of the instant invention are for the treatment/inhibition/prevention of reactive oxygen species (ROS)-related diseases. Elevated levels of reactive oxygen species (ROS), including superoxide, hydroxyl radical, and hydrogen peroxide (H₂O₂) have been associated with the pathogenesis of numerous diseases, such as hypertension, heart failure, arthritis, cancer, neurodegenerative disorders, and cardiovascular diseases (e.g., angiotensin-II induced cardiovascular diseases). The instant invention encompasses methods of inhibiting, treating, and/or preventing oxidative stress associated diseases or disorders (caused by reactive oxygen species (ROS)) comprising the administration of at least one composition of the instant invention to a subject in need thereof. In a particular embodiment, the oxidative stress associated disease or disorder is selected from the group consisting of atherosclerosis, ischemia/reperfusion injury, stroke, traumatic brain injury, brain tumors, stroke, heart attack, meningitis, viral encephalitis, restenosis, hypertension (including in chronic heart failure), heart failure, cardiovascular diseases, cancer, inflammation (e.g., lung inflammation associated with influenza infection), autoimmune disease, an inflammatory disease or disorder, an acute respiratory distress syndrome (ARDS), asthma, inflammatory bowel disease (IBD), a dermal and/or ocular inflammation, arthritis, metabolic disease or disorder, obesity, diabetes, neurological disorders and other disorders of the central nervous system, multiple sclerosis, cerebral palsy, HIV-associated dementia, neurocardiovascular disease/dysregualtion, and neurodegenerative disease or disorder (e.g., Alzheimer's disease, Huntington's disease, Parkinson's disease, Lewy Body disease, amyotrophic lateral sclerosis, and prion disease).

In a particular embodiment, the protein of the nanozyme is superoxide dismutase (SOD; particularly copper zinc SOD or SOD1) and/or catalase. For simplicity, the nanozyme is referred to throughout the application as containing SOD, but the nanozymes may contain catalase. The antioxidant enzyme superoxide dismutase (SOD), particularly, SOD1 (also called Cu/Zn SOD) are known to catalyze the dismutation of superoxide (O₂.⁻). Thus, SOD, particularly SOD1, can be used in antioxidant therapy. It is demonstrated herein that nanozymes containing SOD improve SOD delivery to the brain and provide therapeutic effects. The SOD containing nanozyme may be administered to a subject (e.g., in a composition comprising at least one pharmaceutically acceptable carrier) in order to treat/inhibit/prevent an oxidative stress associated diseases/disorders or reactive oxygen species (ROS)-related disease as described above (e.g., inflammation, neurodegeneration, neurological disorders and other disorders of the central nervous system (including, but not limited to, Alzheimer's disease, Parkinson's disease, neurocardiovascular disease/dysregulation)). In a particular embodiment, the SOD containing nanozyme is administered to a subject in need thereof to treat/inhibit/prevent a neurodegenerative disease (e.g., Alzheimer's disease, Parkinson's disease, Lewy Body disease, amyotrophic lateral sclerosis, and prion disease). In a particular embodiment, the disease is stroke, traumatic brain injury, or hypertension (including in chronic heart failure).

In a particular embodiment, the methods of the instant invention comprise the administration of at least one nanozyme comprising SOD and at least one nanozyme comprising catalase. The SOD (e.g., SOD1) and catalase nanozymes may be administered as a singular composition (e.g., with at least one pharmaceutically acceptable carrier) or administered in separate compositions (e.g., with each composition having at least one pharmaceutically acceptable composition). When the compositions are separate, the SOD and catalase nanozymes may be administered sequentially or simultaneously.

Notably, certain of the above diseases or disorders are more effectively treated with early administration of the nanozyme of the instant invention. In other words, certain of the above diseases/disorders have a preferred therapeutic window for the administration of the nanozyme. For example, in the situation of a stroke or traumatic brain injury, it is desirable to administer the therapeutic agent immediately or soon after the event. In a particular embodiment, the nanozyme is administered within 1 day, within 12 hours, within 6 hours, within 3 hours, or within 1 hour of the event (e.g., stroke or injury).

### III. ADMINISTRATION

The nanozymes described herein will generally be administered to a patient as a pharmaceutical preparation. The term “patient” as used herein refers to human or animal subjects. These nanozymes may be employed therapeutically, under the guidance of a physician or other healthcare professional.

The pharmaceutical preparation comprising the nanozymes of the invention may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of nanozymes in the chosen medium will depend on the hydrophobic or hydrophilic nature of the medium, as well as the size, enzyme activity, and other properties of the nanozymes. Solubility limits may be easily determined by one skilled in the art.

As used herein, “pharmaceutically acceptable medium” or “carrier” includes any and all solvents, dispersion media and the like which may be appropriate for the desired route of administration of the pharmaceutical preparation, as exemplified in the preceding discussion. The use of such media for pharmaceutically active substances is known in the art. Except insofar as any conventional media or agent is incompatible with the nanozyme to be administered, its use in the pharmaceutical preparation is contemplated.

The dose and dosage regimen of a nanozyme according to the invention that is suitable for administration to a particular patient may be determined by a physician considering the patient's age, sex, weight, general medical condition, and the specific condition for which the nanozyme is being administered and the severity thereof. The physician may also take into account the route of administration of the nanozyme, the pharmaceutical carrier with which the nanozyme is to combined, and the nanozyme's biological activity.

Selection of a suitable pharmaceutical preparation will also depend upon the mode of administration chosen. For example, the nanozymes of the invention may be administered by direct injection into an area proximal to the BBB or intravenously. In these instances, the pharmaceutical preparation comprises the nanozymes dispersed in a medium that is compatible with the site of injection.

Nanozymes may be administered by any method such as intravenous injection or intracarotid infusion into the blood stream, intranasal administration, oral administration, or by subcutaneous, intramuscular or intraperitoneal injection. Pharmaceutical preparations for injection are known in the art. If injection is selected as a method for administering the nanozymes, steps must be taken to ensure that sufficient amounts of the molecules reach their target cells to exert a biological effect. The lipophilicity of the nanozymes, or the pharmaceutical preparation in which they are delivered, may have to be increased so that the molecules can arrive at their target location. Furthermore, the nanozymes may have to be delivered in a cell-targeting carrier so that sufficient numbers of molecules will reach the target cells. Methods for increasing the lipophilicity of a molecule are known in the art.

Pharmaceutical compositions containing a nanozyme of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, intranasal, oral, direct injection, intracranial, and intravitreal. In preparing the nanozyme in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which solid pharmaceutical carriers are employed. If desired, tablets may be sugar-coated or enteric-coated by standard techniques. Injectable suspensions may also be prepared, in which case appropriate liquid carriers, suspending agents and the like may be employed. Additionally, the nanozyme of the instant invention may be administered in a slow-release matrix. For example, the nanozyme may be administered in a gel comprising unconjugated poloxamers.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of nanozymes may be determined by evaluating the toxicity of the molecules in animal models. Various concentrations of nanozyme pharmaceutical preparations may be administered to mice, and the minimal and maximal dosages may be determined based on the beneficial results and side effects observed as a result of the treatment. Appropriate dosage unit may also be determined by assessing the efficacy of the nanozymes treatment in combination with other standard drugs. The dosage units of nanozymes may be determined individually or in combination with each treatment according to the effect detected.

The pharmaceutical preparation comprising the nanozymes may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The following example provides illustrative methods of practicing the instant invention, and is not intended to limit the scope of the invention in any way.

### EXAMPLE

### Materials and Methods

### Materials

SOD1 (from bovine erythrocytes), hydrogen peroxide (H₂O₂), 2,3,5-triphenyltetrazolium chloride (TTC) and copper standards for Inductively Coupled Plasma Mass Spectroscopy (ICP-MS)—TraceCERT®, 1000 mg/L Cu in nitric acid) were from Sigma-Aldrich (St. Louis, Mo.). Catalase (from bovine liver) was from Calbiochem (Gibbstown, N.J.). PEG-pLL₅₀ was from Alamanda Polymers™ (Huntsville, Ala.). Its molecular mass determined by gel permeation chromatography was 13,000 Da and polydispersity index was 1.09; the PEG molecular mass was 4600 Da and the degree of polymerization of pLL block was 51. Cross-linkers 3,3′-dithiobis(sulfosuccinimidylpropionate) (DTSSP) and bis(sulfosuccinimidyl)suberate (BS³) were from Thermo Fisher Scientific (Rockford, Ill.). NAP™ desalting columns and HiPrep 16/60 Sephacryl S-400 HR column were from GE Healthcare (Piscataway, N.J.). Criterion Tris-HCl gels and Precision Plus Protein™ All Blue Standards were from Bio-Rad (Hercules, Calif.). SYPRO® Ruby protein gel stain and cell culture reagents were purchased from Invitrogen (Carlsbad, Calif.). CellTiter 96® AQueous One Solution Cell Proliferation Assay (MTS) was from Promega (Madison, Wis.). LumiMax Superoxide Anion Detection Kit was from Agilent Technologies, Inc. (Santa Clara, Calif.). All other reagents and supplies were from Fisher Scientific (Pittsburgh, Pa.) unless noted otherwise.

### Synthesis and Purification of Cl-Nanozymes

Enzyme and polymer stock solutions were prepared in 10 mM HEPES (pH 7.4) and 10 mM HEPES-buffered saline (HBS; pH 7.4) for SOD1 and catalase, respectively. Non-cross-linked BICs (Z+/−=2) were prepared as described (Klyachko et al. (2012) Nanomed. Nanotechnol. Biol. Med., 8:119-129). Targeted degree of cross-linking was defined as the molar ratio between cross-linker (DTSSP/BS³) and pLL amines. Precalculated amount of the respective cross-linker was dissolved in the reaction buffer (HEPES/HBS), quickly added to BICs, and the reaction mixture was briefly vortexed and incubated for 2 hours on ice. Unreacted crosslinker was desalted using NAP™ columns following manufacturer's instructions. Cl-Nanozymes were purified using size exclusion chromatography (SEC) (small/intermediate scale) or centrifugal filtration (large scale). SEC was carried out using an ÄKTA™ Fast Protein Liquid Chromatography (FPLC) (Amersham Biosciences, Piscataway, N.J.) system. DTSSP cl-nanozymes were lyophilized overnight, reconstituted in deionized water (DW), loaded onto a HiPrep 16/60 Sephacryl™ S-400 HR column and eluted using 10 mM HBS (pH 7.4) at a flow rate of 0.5 mL/minute. Prior to each experiment, the column was pre-conditioned with free block copolymer (PEG-pLL50) to minimize non-specific adsorption of the cl-nanozymes (Boeckle et al. (2004) J. Gene Med., 6:1102-1111). Fractions spanning each distinct peak were pooled, concentrated using Amicon® Ultra-4 Centrifugal Filter Units with a molecular weight cutoff (MWCO) of 3000 Da and desalted using NAP™ columns as needed. Protein concentration was determined using Micro BCA™ Protein Assay Kit. In large-scale preparations, clnanozymes were purified by centrifugal filtration using Macrosep™ Centrifugal Device (Pall Life Sciences, Ann Arbor, Mich.) with a MWCO of either 100 kDa (for SOD1) or 1000 kDa (for catalase). Briefly, unreacted cross-linker in cross-linked BICs was desalted using NAP™ columns and eluate was collected in 10 mM HEPES containing 0.3 M NaCl (pH 7.4). Samples were loaded onto the centrifugal device and concentrated to 10% original volume by centrifuging at 4500 RPM. Two rounds of purification were done in 10 mM HEPES buffer containing 0.3 M NaCl (pH 7.4) and the third round was done in 10 mM HEPES buffer (pH 7.4). The concentrate was collected and desalted using NAP™ columns to remove excess NaCl.

### Dynamic Light Scattering (DLS)

Intensity-mean z-averaged particle diameter (Deff), polydispersity index (PDI), and ζ-potential were measured using a Zetasizer Nano ZS (Malvern Instruments Ltd, MA) (Klyachko et al. (2012) Nanomed. Nanotechnol. Biol. Med., 8:119-129). Both size and ζ-potential measurements were conducted in low ionic strength buffer (10 mM HEPES, pH 7.4) unless indicated otherwise. Wherever indicated, catalase BICs were desalted to remove excess NaCl before measuring ζ-potential. Data is represented as mean values (n=3).

### ICP-MS

Copper (Cu²⁺) content in SOD1 samples was determined using ICP-MS. Standards/samples were diluted in double distilled nitric acid and measurements were performed in 10 replicates using a PerkinElmer Nexion 300Q ICP Mass Spectrometer. The data were analyzed using the Total Quantity method. Concentration of the predominant isotope, ⁶³C was calculated from the standard curve generated using copper standards.

### Enzyme Activity

SOD1 enzyme activity was determined using two independent methods —inhibition of PG autoxidation by added SOD1 (indirect method) (Yi et al. (2010) Free Radic. Biol. Med., 49:548-58) and scavenging of experimentally generated superoxide radicals (O₂.⁻) by added SOD1 using Electron Paramagnetic Resonance (EPR) spectroscopy (direct method) (Rosenbaugh et al. (2010) Biomaterials, 31:5218-5226). Wherever indicated, SOD1 activity measured using PG assay was normalized to Cu²⁺ content determined using ICP-MS. Enzyme activity of catalase was measured by following decomposition of H2O2 (Li et al. (2007) J. Biomol. Tech., 18:185-187). Slope (reaction rate of H₂O₂ decomposition) was calculated as ΔA240/minute. Catalytic activity of catalase among the different samples was compared in terms of the slope of linear regression. Activity was expressed as percent (%) relative to native enzyme. Enzyme activity of SOD1 (reported by Sigma-Aldrich) was ˜4,000 U/mg protein and catalase (reported by Calbiochem) was ˜46,500 U/mg protein.

### Gel Retardation Assay

Formation of cl-nanozymes was confirmed by their compromised ability to migrate in a polyacrylamide gel under denaturing conditions. Five or 3 μg protein (SOD1 or catalase) was denatured in the sample buffer (no reducing agent added), loaded on a 18/10% Criterion Tris-HCl gel and electrophoresed at 200 V (100 mA) for 1 hour, and stained using SYPRO® Ruby protein gel stain and imaged on a Typhoon gel scanner (Amersham Biosciences Corporation, Piscataway, N.J.) at 100μ pixel size.

### Sedimentation Equilibrium Analysis

Molecular weight of purified SOD1 cl-nanozymes was determined by sedimentation equilibrium analysis using a Beckman Optima XL-I analytical ultracentrifuge and an AN-60Ti rotor (Sorgen et al. (2004) Biophys. J., 87:574-581). Data analysis was performed using the Beckman XLA/XL-I software package with Microcal, ORIGIN v4 software.

### Cell Culture

Immortalized bovine brain microvessel endothelial cells containing a Middle T-antigen gene (TBMECs) and CATH.a neuronal cell line (CRL-11179™) were from American Type Culture Collection (Manassas, Va.) and were cultured as described (Rosenbaugh et al. (2010) Biomaterials 31:5218-5226; Yazdanian et al., Immortalized Brain Endothelial Cells in, Boehringer Ingelheim Pharmaceuticals, Inc., Ridgefield, Conn., 2000).

### Cytotoxicity Assay

TBMECs were seeded at 5,000 cells/well in a collagen, fibronectin-coated 96 well plate and cultured until 100% confluence. CATH.a cells were seeded at 10,000 cells/well and differentiated into neurons as described above. Cells were incubated with indicated concentrations of samples (in case of the free polymer control, cells were treated with equivalent concentrations of PEG-pLL50 that would be present in non-cross-linked BICs) diluted in complete growth medium for 24 hours and cell viability was determined using a commercially available MTS assay kit. Percent (%) cell viability was calculated using the formula=(Aₛₐₘₚₗₑ/A_{untreated} cells)×100. Data represents average±SD (n=3).

### O₂.⁻ Scavenging In Vitro in Cell Cultures

TBMECs/CATH.a cells were grown in 48 well plates and were treated with SOD1/formulated SOD1 (50 μg/mL) diluted in complete growth medium for 2 hours. Treatment mixture was removed and cells were further incubated with fresh medium for different times: 0, 1, 2, 4 or 12 hours. Post-incubation, cells were washed using PBS and lysed using 1× cell lysis buffer (Cell Signaling Technology, Boston, Mass.). Hypoxanthine and xanthine oxidase were used to generate O₂.⁻ in cell lysates and LumiMax Superoxide Anion Detection Kit was used to determine percent (%) O₂.⁻ remaining (relative to untreated cells). Data represent average±SD (n=4).

### Rat MCAO Model and Experimental Details

Young adult male Sprague-Dawley rats (250-300 g) were anesthetized with ketamine/xylazine cocktail and isoflurane. Right common carotid artery was incised, and a filament with bulbous tip was inserted through this incision into internal carotid artery and further until bifurcation of middle cerebral artery (MCA). Bulbous tip occluded the entrance to MCA and blocked blood supply to part of the right brain hemisphere of the rat. Filament was carefully withdrawn after 2 hours and immediately incision on MCA was permanently closed and 0.5 mL saline, native SOD1 or purified SOD1 cl-nanozyme was administered i.v. via the tail vein at a dose of 10 kU/kg body weight. Post-surgery rats were returned to their cages for 22 hours. Sensorimotor functions of rats (response to touch of a side of a trunk, touch of vibrissae on one side, forelimbs outreach, floor walking and climbing of a cage wall) were evaluated 24 hours after the beginning of ischemic episode (Sun et al. (2008) Brain Res., 1194:73-80). After the evaluation, rats were euthanized and brains were dissected. Dissected brains were sectioned (6 sections 2 mm thick each) and sections were stained with TTC to visualize the infarct region. Stained sections were photographed and digital images were quantified using ImageJ software (National Institute of Health, Bethesda, Md.). Infarct areas were outlined and determined (in conditional units) as follows: [(infarct area #1)/(entire hemisphere area #1)+ . . . . (infarct area #6)/(entire hemisphere area #6)]=infarct index of brain #1. Data is represented as infarct index average (5-6 animals/group)±standard error of mean (SEM).

### Statistical Analysis

Statistical comparisons between two groups were made using Studen's t-test while comparisons between multiple groups were done using non-parametric one-way ANOVA with multiple comparisons (Kruskal-Wallis) using Origin 8.5 software (Northampton, Mass.). P-value<0.05 was considered statistically significant.

### Determination of SOD1 Activity Using EPR Method

SOD1 activity was measured using EPR method as described (Rosenbaugh et al. (2010) Biomaterials 31:5218-5226). Briefly, SOD1 or SOD1-containing sample was added to a mixture containing the EPR probe, 1-Hydroxy-3-methoxycarbonyl-2,2,5,5-tetramethylpyrrolidine (CMH) (200 μM), hypoxanthine (25 μM), and xanthine oxidase (10 mU/mL) in 100 μL Krebs-HEPES buffer. Fifty μL of the sample was loaded into a glass capillary tube and was inserted into the capillary holder of a Bruker e-scan EPR spectrometer. A range of concentrations (1.4×10⁻⁵ to 1.4×10⁻² ppm Cu²⁺) of SOD1 or SOD1-containing sample was used to construct a dose-response plot and percent (%) inhibition of the CMH-O₂.⁻ signal by added SOD1 was calculated relative to the sample that contained no SOD1. O₂.⁻ scavenging capability of SOD1 among the different samples was compared in terms of an IC50 value, defined as the concentration of SOD1 that resulted in 50% inhibition of the CMH-O₂.⁻ signal.

### Determination of Catalytic Constants

Catalytic constants (turnover number; kcat and Michaelis constant; Km) for catalase samples were determined by studying the effect of substrate concentration (H₂O₂) on reaction rate of H₂O₂ decomposition as described (Klyachko et al. (2012) Nanomed. Nanotech. Biol. Med., 8:119-129). Briefly, catalase (2 μg/mL) was added to a cuvette containing substrate (H₂O₂) in concentrations ranging from 0 to 65 mM in 50 mM phosphate buffer (pH 7.4). Decomposition of H₂O₂ was followed at 240 nm for 1 minute and reaction rate was calculated from the slope (ΔA240/minute). Catalytic constants were determined from double reciprocal Lineweaver-Burk plots depicting effect of substrate concentration (1/[S]) on reaction rate (1/slope).

### Atomic Force Microscopy (AFM)

Tapping mode AFM imaging was performed in air as described (Kim et al. (2010) Biomacromolecules 11:919-926). Briefly, 7 μL of a 0.5 mg/mL sample was deposited on an aminopropylytriethoxy silane (APS) mica surface (Lyubchenko et al. (2009) Methods Mol. Biol., 543:337-351; Shlyakhtenko et al. (2003) Ultramicroscopy 97:279-287) for 2 minutes, washed with water and dried under argon atmosphere. AFM imaging was performed using a Multimode NanoScope IV system (Veeco, Santa Barbara, Calif.) operated in tapping mode.

### Synthesis of DTBP Cross-Linked Nanozymes

Non-cross-linked BICs were prepared (Klyachko et al. (2012) Nanomed. Nanotech. Biol. Med., 8:119-129) and DTBP cross-linking was carried out as reported (Oupicky et al. (2001) Gene Ther., 8:713-724).

### Results

### Synthesis, Characterization and Purification of Cl-Nanozymes

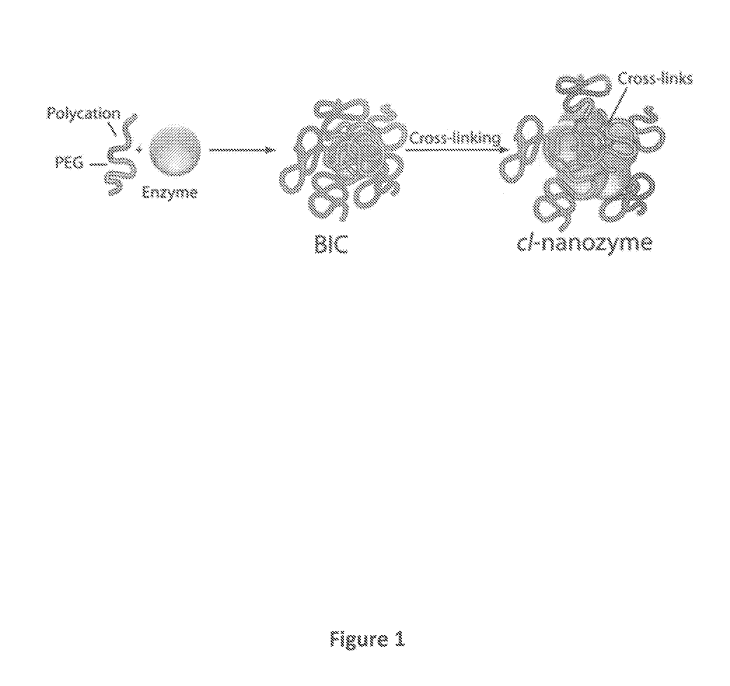

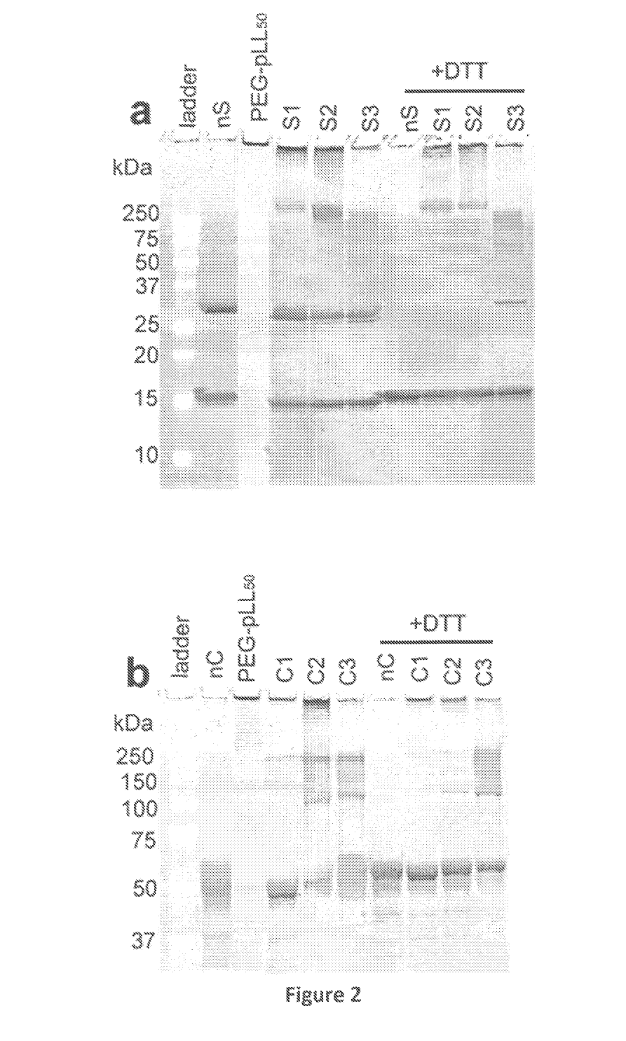

cl-Nanozymes containing either SOD1 or catalase were synthesized. A graphical representation of the cl-nanozymes is provided in FIG. 1. Samples are denoted as follows throughout: nS or nC—native SOD1 or catalase; S1 or C1—BICs of SOD1 or catalase (Z=2), S2 (S2ₚ) or C2 (C2ₚ)—DTSSP-cross-linked cl-nanozymes; and S3 (S3ₚ) or C3 (C3ₚ)—BS³-cross-linked cl-nanozymes. Subscript index “p” refers to purified samples. The targeted degree of cross-linking defined earlier was optimized for each enzyme and cross-linking chemistry to ensure that the enzyme retained at least 75% of the initial activity of the native enzyme. The optimal targeted degrees of cross-linking for SOD1 and catalase cl-nanozymes were determined to be 0.5 and 1.0, respectively. Cross-linking was confirmed by retarded enzyme migration in denaturing gel electrophoresis (FIG. 2). DTSSP produced cross-links that contained cleavable disulfide bonds, while BS³ produced non-cleavable cross-links. Dithiothreitol (DTT) treatment cleaved disulfide cross-links, noted as a decrease in the high molecular mass band density corresponding to DTSSP-cl-nanozymes (FIG. 2). In contrast, there were no changes in band density in the case of BS³-cl-nanozymes with non-cleavable cross-links.

The physicochemical characteristics (hydrodynamic diameters, PDI, ζ-potential) and enzyme activity of samples are listed in Table 1. The values of D_{eff} for native SOD1 and catalase were in good agreement with the theoretical hydrodynamic diameters estimated using the Protein Utilities module in the Malvern Zetasizer Nano software (5.2 and 12.5 nm for SOD1 and catalase, respectively). In the SOD1 formulations, there was nearly 2-fold increase in the particle size upon BIC formation, accompanied by a change in the ζ-potential from weakly-negative (native enzyme) to a positive value. The positive ζ-potential of this BIC could be due to some excess of amino groups of either the protein or pLL incorporated into the complex. Notably the size measurements in this case were carried out in low ionic strength buffer, since addition of 0.15 M NaCl favored dissociation of BICs, as noted by the decrease in the particle size. The size increased further three-fold after the BICs were cross-linked suggesting that such cl-nanozymes contained multiple SOD1 protein molecules. Interestingly, after the cross-linking the ζ-potential decreased and again became weakly negative, which may be indicative of consumption of the protein and/or pLL amino groups that reacted with the cross-linking reagents. The size measurements with catalase BIC were quite interesting in comparison to those of SOD1. Here the sizes of BIC practically did not change compared to the free catalase suggesting that the BIC contained only one catalase protein molecule. However, after cross-linking the sizes increased by about 3.7-fold indicating that multiple catalase molecules were assembled in the cl-nanozymes. The ζ-potential of the catalase or its BIC was not directly measured since they were not stable at low ionic strength and were prepared in 10 mM HBS buffer, pH 7.4, containing 0.15 M NaCl. However, the pre-formed BIC was rapidly desalted and its ζ-potential was determined, which was positive. This was in contrast to the native catalase that was negative, suggesting that BICs were indeed formed notwithstanding the lack of the size changes. Finally, similar to the previous case the ζ-potential of cl-nanozymes was lower (and negative) than that of the non-cross-linked BIC.

TABLE 1
Characteristics of BICs and cl-nanozymes.

| | | | | Enzyme activity, |
|---|---|---|---|---|
| Sample^{a,b} | D_{eff}, nm | PDI^{c} | ζ-potential, mV | % of initial |
| nS | 5.2 | 0.10 | −1.3 | 100 |
| S1^{d} | 9.8 | 0.14 | +7.8 | 97 |
| S2 | 31.0 | 0.13 | −0.1 | 91 |
| S3 | 29.8 | 0.20 | −1.9 | 77 |
| nC | 14.9 | 0.40 | −16.3^{e} | 100 |
| C1^{d} | 13.1 | 0.26 | +16.3^{e} | 121 |
| C2 | 55.6 | 0.28 | −11.0^{e} | 100 |
| C3^{f} | n.a. | n.a. | n.a. | n.a. |

^{a}nS or nC—native SOD1 or catalase; S1 or C1—non-cross-linked BICs of SOD1 or catalase, S2 or C2—cleavable (DTSSP cross-linked) cl-nanozymes, and S3 or C3—non-cleavable (BS³ cross-linked) cl-nanozymes;
^{b}All SOD1-containing samples (1 mg/mL) were in low ionic strength buffer, 10 mM HEPES, pH 7.4, while the catalase samples, 0.5 mg/mL were in 10 mM HBS, pH 7.4 (unless noted otherwise);
^{c}polydispersity index;
^{d}Z = 2;
^{e}the measurements were carried out in 10 mM HEPES, pH 7.4 after desalting the samples;
^{f}not available since BS³ did not cross-link the catalase BICs.

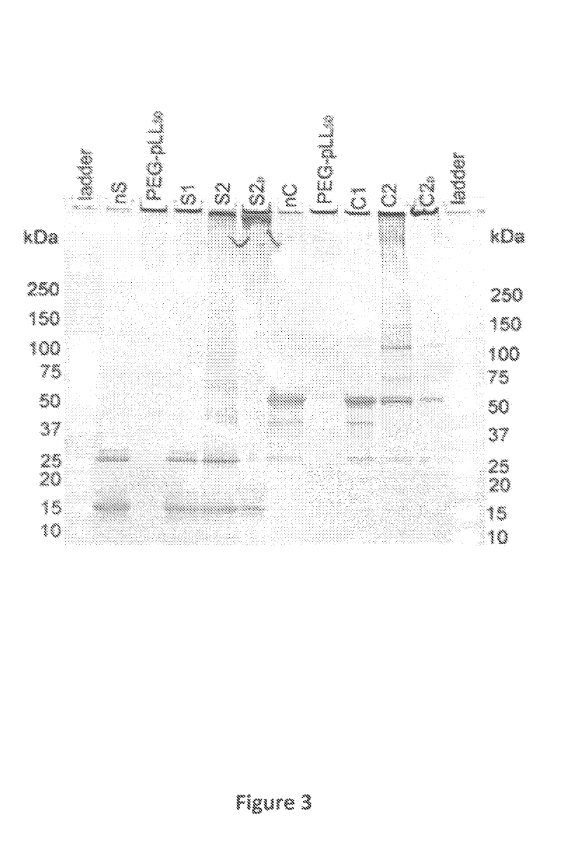

Denaturing gel electrophoresis (FIGS. 2 and 3) showed that the cross-linked samples contained considerable amounts of free enzymes (two main bands corresponding to the monomer (16 kDa) and dimer (32 kDa) forms of SOD1 and several bands corresponding to the monomer (62 kDa), dimer (124 kDa) and tetramer (247 kDa) forms of catalase). The free PEG-pLL₅₀ may have also been present in these samples as it did not enter the gel and remained in the wells similar to the cl-nanozymes (SYPRO® Ruby also stains basic amino acids like lysines). As it follows the sample, homogeneity was improved by SEC purification. After purification, almost the entire sample (S2p and C2p) remained in or near the wells, while only a minor portion of proteins (mainly their respective monomers) migrated through the polyacrylamide gel (FIG. 3).

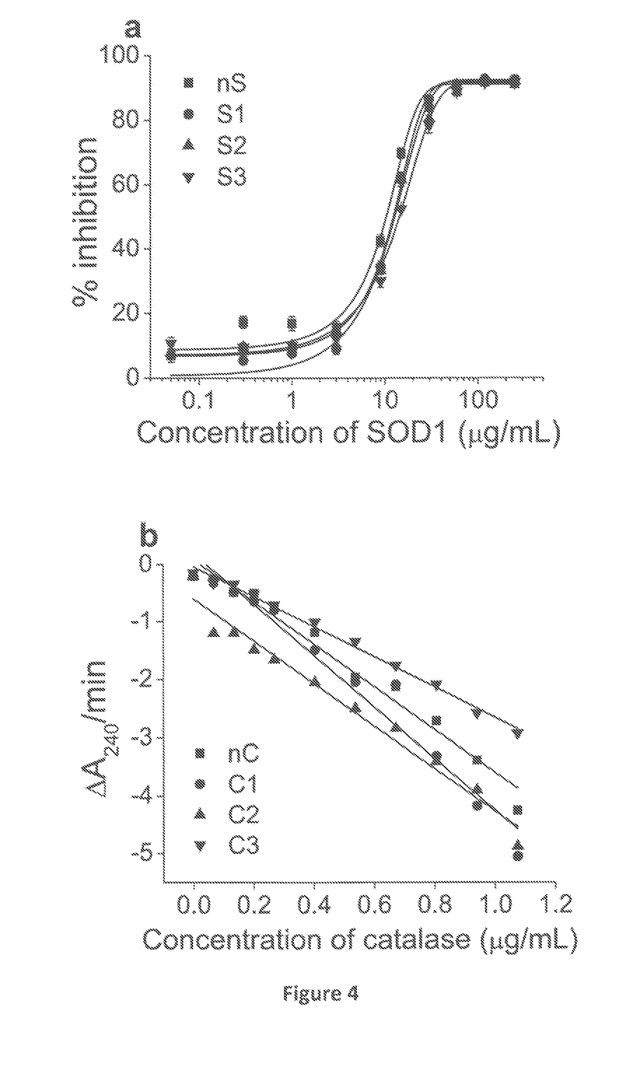

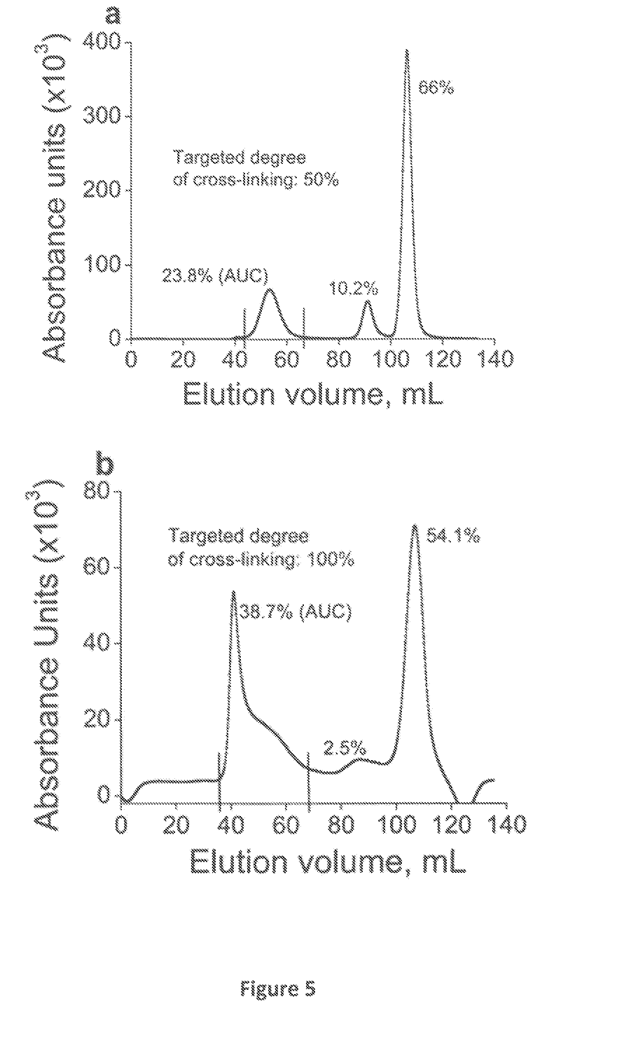

The catalytic activity of SOD1 was determined by following inhibition of PG autoxidation by SOD1 (Marklund et al. (1974) Eur. J. Biochem., 47:469-474) and a typical dose response curve is shown in FIG. 4A Inhibitory effect of SOD1 on PG autoxidation among the different samples was compared in terms of an IC₅₀ value, defined as the concentration of SOD1 that inhibited PG autoxidation by 50% (Yi et al. (2010) Free Radic. Biol. Med., 49:548-58). Catalytic activity of catalase was determined using the standard H₂O₂ decomposition assay (Li et al. (2007) J. Biomol. Tech., 18:185-187) and a typical dose response plot is shown in FIG. 4B. Both non-cross linked BICs (S1 or C1) and cl-nanozymes (e.g., S2, S3, and C2) retained relatively high activities (77-100%) of the unmodified enzyme.

### Purification and Further Characterization of Cl-Nanozymes

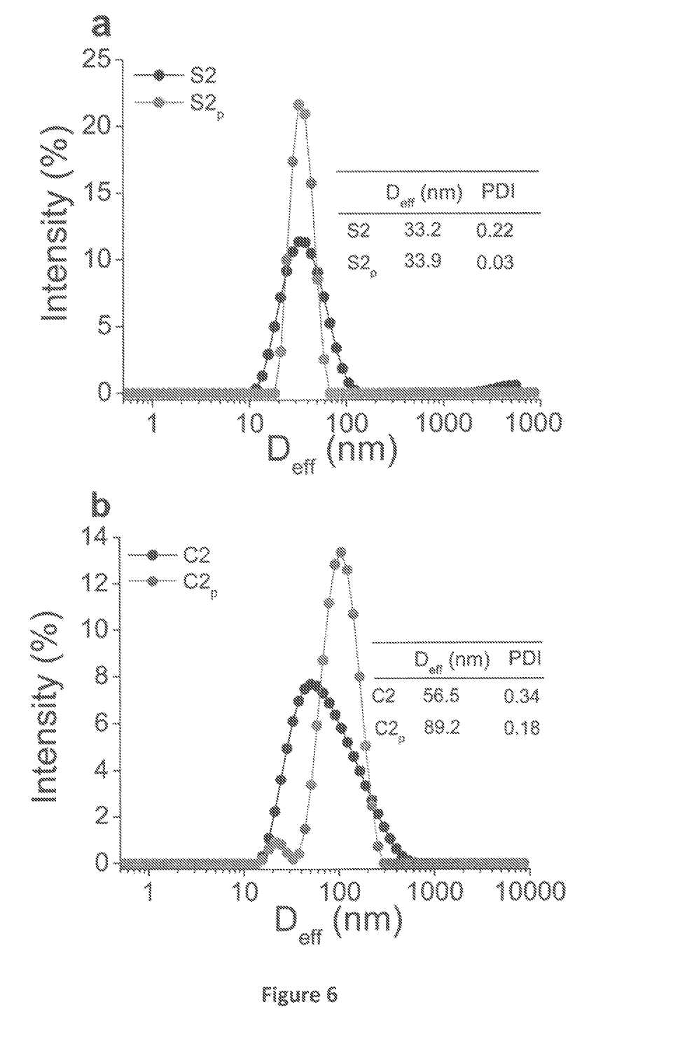

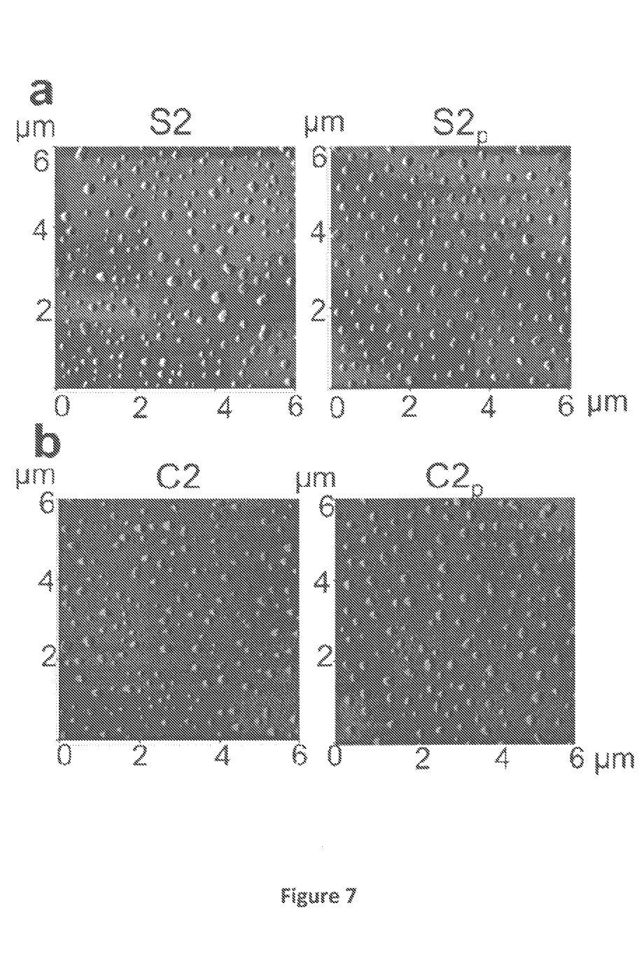

Since sample homogeneity is crucial for the pharmaceutical protein formulations, the cl-nanozymes (S2, C2) were purified by separating them from the non-cross linked BIC components using SEC (FIG. 5). Based on the area-under-the-curve (AUC) analysis the cross-linked particles comprised ca. 24% and 39% of the non-purified samples of SOD1 and catalase cl-nanozymes, respectively. The rest was mostly the free enzyme (nS, nC) and a minor portion of non-cross-linked BIC (S1, C1) that did not dissociate during chromatography. The elution volumes of the three fractions —cl-nanozymes (C2p 41 mL, S2p 53 mL), noncross-linked BICs (C1 87 mL, S1 91 mL) and free enzymes (nC 107 mL, nS 106 mL) were in logical agreement with the respective particle sizes (Table 1). Particle sizes measured using DLS demonstrated that after purification the D_{eff} of SOD 1 cl-nanozyme practically did not change while the D_{eff} of catalase cl-nanozyme increased ca. 60% (FIG. 6). Incidentally the PDI of both samples considerably decreased. The purified cl-nanozymes (S2p and C2p) retained their spherical morphology observed under AFM (FIG. 7), albeit they were more uniform compared to nonpurified cl-nanozymes (S2 and C2).

Table 2 lists the enzyme activity retained by purified cl-nanozymes. SOD1 concentration in cl-nanozyme samples was normalized to Cu²⁺ content determined by ICP-MS, and the activity was assayed by PG autoxidation as described before. After purification this cl-nanozyme (S2p) retained only ca. 47% of the activity of the non-purified cl-nanozyme (S2) and native SOD1 (nS) samples. This result was generally consistent with the SOD1 activity measurements using EPR spectroscopy (Table 3) although EPR results showed slightly higher activity for S2p. However, the purified catalase cl-nanozyme (C2p) was nearly as active as the non-purified cl-nanozyme (C2) and native catalase (nC) in H₂O₂ decomposition assay. A more detailed analysis, however, indicated that the purified cl-nanozyme (C2p) had somewhat lower k_{cat} compared to non-purified sample (C2), albeit the value was nearly similar to native catalase (nC) (Table 4). However, the increase in the k_{cat} of non-purified cl-nanozyme (C2) compared to native catalase was offset by ca. 1.8-fold increase in its Kₘ value. As a result the catalytic efficiency (k_{cat}/Kₘ) of the non-purified cl-nanozyme (C2) and native enzyme (nC) were nearly the same whereas the k_{cat}/Kₘ of purified cl-nanozyme (C2p) was ca. 48 and 53% lower than nC and C2, respectively.

TABLE 2
Enzyme activity of purified cl-nanozymes.

| | Enzyme Activity |
|---|---|
| Sample | % of Initial |
| S2 | 96 |
| S2ₚ | 45 |
| C2 | 100 |
| C2ₚ | 100 |

S2 or C2—cleavable (DTSSP cross-linked) cl-nanozymes containing SOD1 or catalase and subscript ‘p’ refers to their respective purified forms.

TABLE 3
Enzyme activity of purified SOD1 cl-nanozymes determined by EPR.

| | Enzyme Activity |
|---|---|
| Sample^{a} | % of Initial |
| S2 | 91 |
| S2ₚ | 57 |

^{a}S2 and S2ₚ refer to cleavable (DTSSP cross-linked) SOD1 cl-nanozymes, subscript ‘p’ refers to its purified form.

TABLE 4
Catalytic constants of cl-catalase nanozymes.

| Sample^{a} | k_{cat}, min⁻¹ | Kₘ, mM | K_{cat}/Kₘ, min⁻¹ mM⁻¹ |
|---|---|---|---|
| nC | 4.79 × 10⁷ | 53.2 | 9.00 × 10⁵ |
| C2 | 9.36 × 10⁷ | 94.1 | 9.95 × 10⁵ |
| C2ₚ | 4.33 × 10⁷ | 93.2 | 4.65 × 10⁵ |

^{a}nC—native catalase, C2 and C2ₚ—cleavable (DTSSP cross-linked) cl-nanozymes; subscript ‘p’ refers to the purified form.

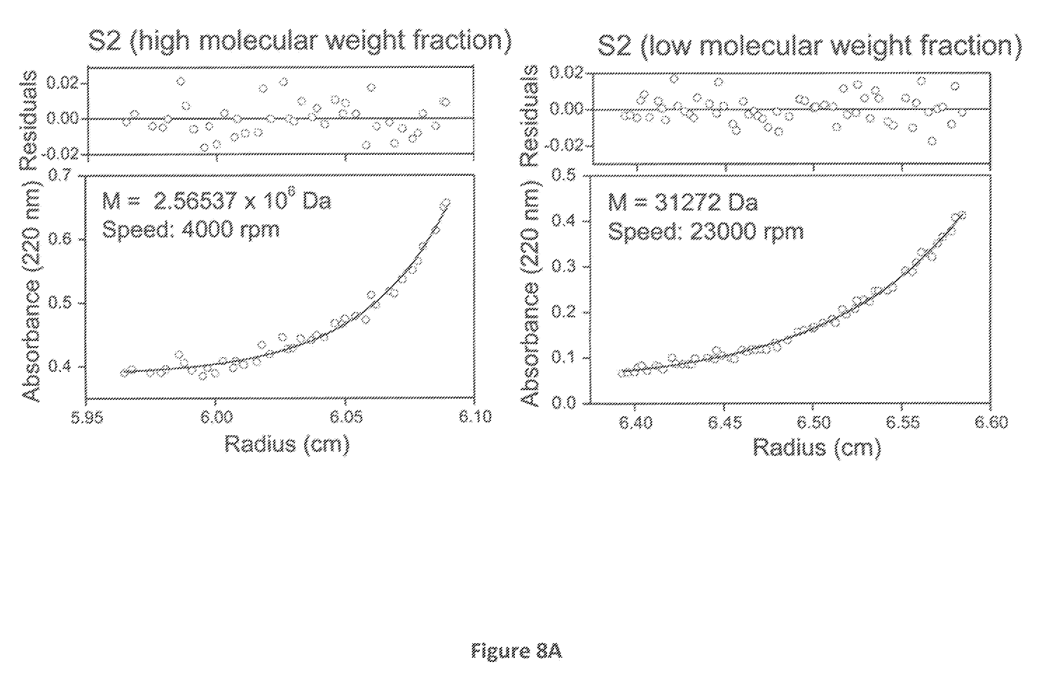

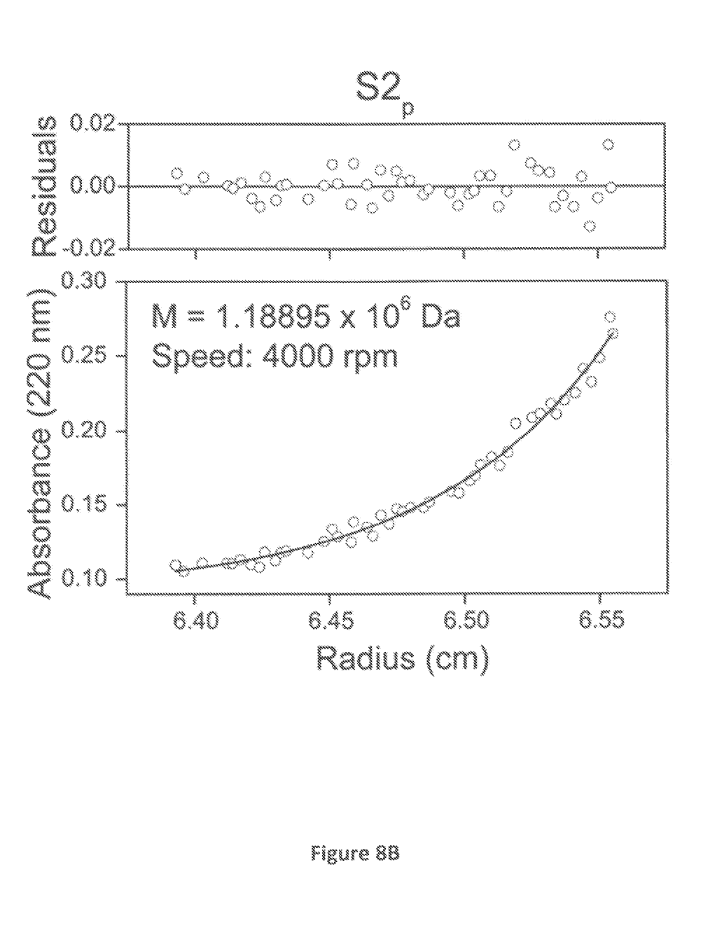

The molecular mass and aggregation state of the SOD1 cl-nanozyme was determined using analytical ultracentrifuge sedimentation equilibrium analysis (FIG. 8). The analysis was carried out in 10 mM HBS, which favors dissociation of the noncross-linked BIC. As expected, this method revealed the presence of mixture of cl-nanozymes and native enzyme in the non-purified sample. The molecular masses determined by this method were in a reasonable agreement with the theoretical estimate of 4.4 MDa for cl-nanozymes (calculated assuming formation of a stoichiometric complex with a D_{eff} of 30 nm) and in excellent agreement with the theoretical value of 32 kDa for native SOD1. Purified cl-nanozymes (S2p) showed an experimental molecular weight of ca. 1.2 MDa, which indicates that they contained ca. 30 SOD1 globules. This observation also points out that the purified sample contained no aggregate(s), which is consistent with the DLS data. Sedimentation equilibrium analysis has a molecular mass range from 2500 Da to 1.5×10⁶ Da; therefore the catalase cl-nanozymes could not be analyzed using this technique.

### In Vitro Studies

In vitro experiments were conducted using two cell line models. TBMEC monolayers were used as an in vitro model of brain microvessel endothelial cells (BMECs). This cell line retains morphological and biochemical features of primary BMECs and has been described as a suitable in vitro model for BBB studies (Yazdanian et al., Immortalized Brain Endothelial Cells in, Boehringer Ingelheim Pharmaceuticals, Inc., Ridgefield, Conn., 2000). CATH.a cells were differentiated into neurons (Rosenbaugh et al. (2010) Biomaterials 31:5218-5226) and were used as a model of central neurons. It should be noted that only SOD1 formulations were used in all studies henceforth.

### Cytotoxicity of Formulations

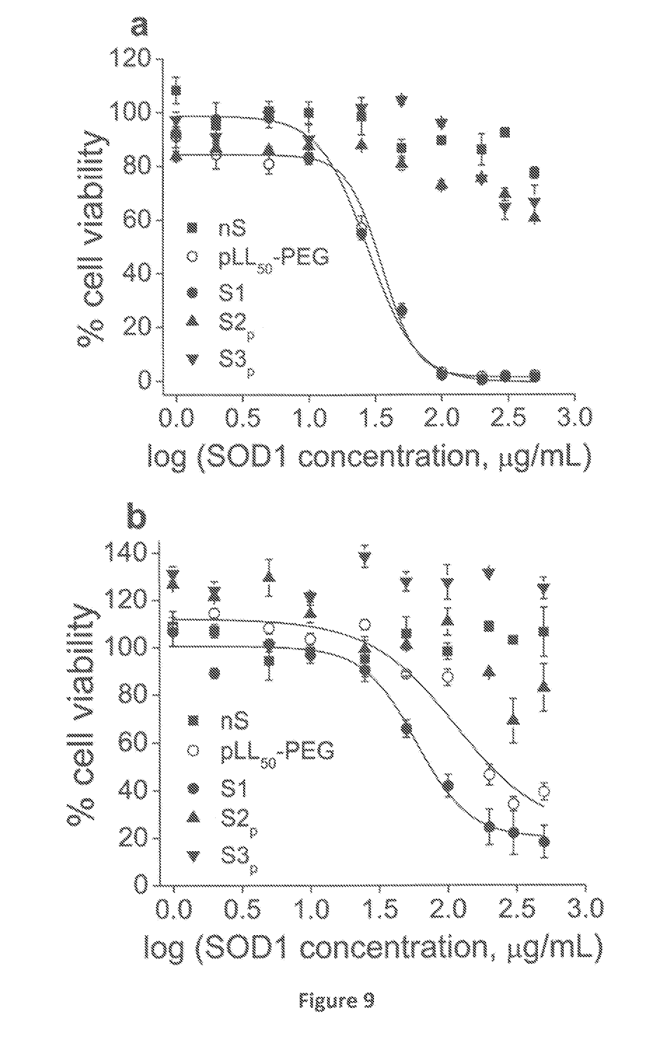

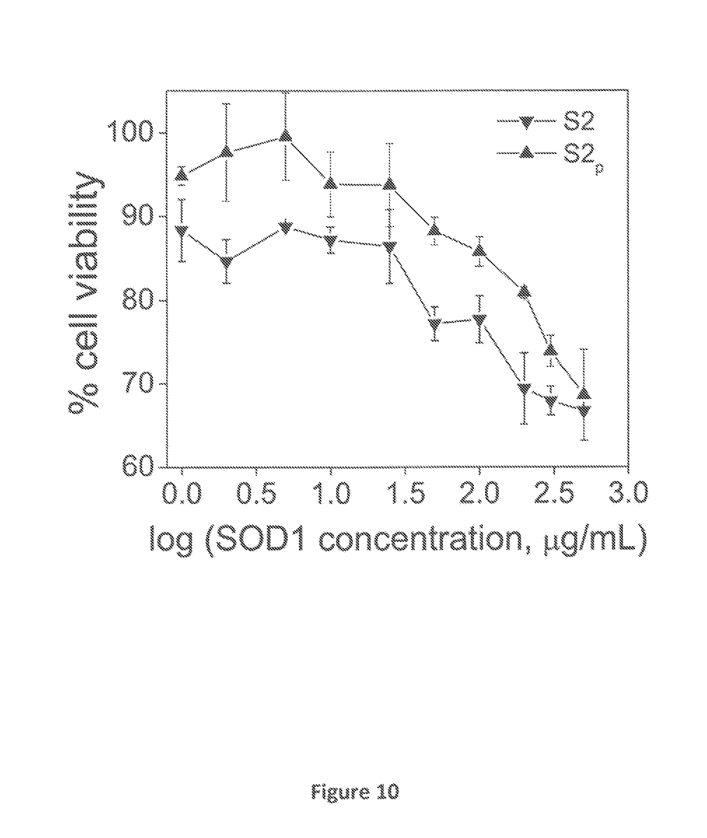

Cytotoxicity of SOD1 formulations was evaluated in both TBMEC monolayers and CATH.a neurons (FIG. 9). The IC₅₀ values of the free block copolymer (PEGpLL₅₀) and non-cross-linked BIC (S1) were ˜35 and 28 μg/mL (TBMEC) and 59 and 113 μg/mL (CATH.a neurons), respectively. This data indicates that the toxicity of non cross-linked BIC (Z=2) may be due to the admixture or PEG-pLL₅₀ release, which interacts with negatively charged cellular membranes and other macromolecules through its polycation chain, a well-documented phenomenon for polycations (Godbey et al. (2001) Biomaterials 22:471-480; Moghimi et al. (2005) Mol. Ther., 11:990-995). In contrast, purified cl-nanozymes (S2p and S3p) displayed significantly lower toxicity with cell viabilities of 60-70% (TBMEC) and 83-100% (CATH.a neurons) at the highest concentration tested (500 μg/mL). Hence, no IC₅₀ value could be determined for purified cl-nanozymes in the tested range of concentrations. In general, cells treated with DTSSP-cl-nanozymes (S2p) at concentrations ≧25 μg/mL showed slightly lower cell viabilities compared to those treated with BS³-cl-nanozymes (S3p). The difference was more pronounced in CATH.a neurons where the cell viability was ca. 26% less for S2p compared to S3p. Notably, the non-purified cl-nanozymes was slightly more toxic than the purified samples (FIG. 10). Therefore, purification and in particular removal of the free PEG-pLL₅₀ is an important factor decreasing cellular toxicity.

### Superoxide Scavenging Capability of SOD1 Formulations in Cultured Cells

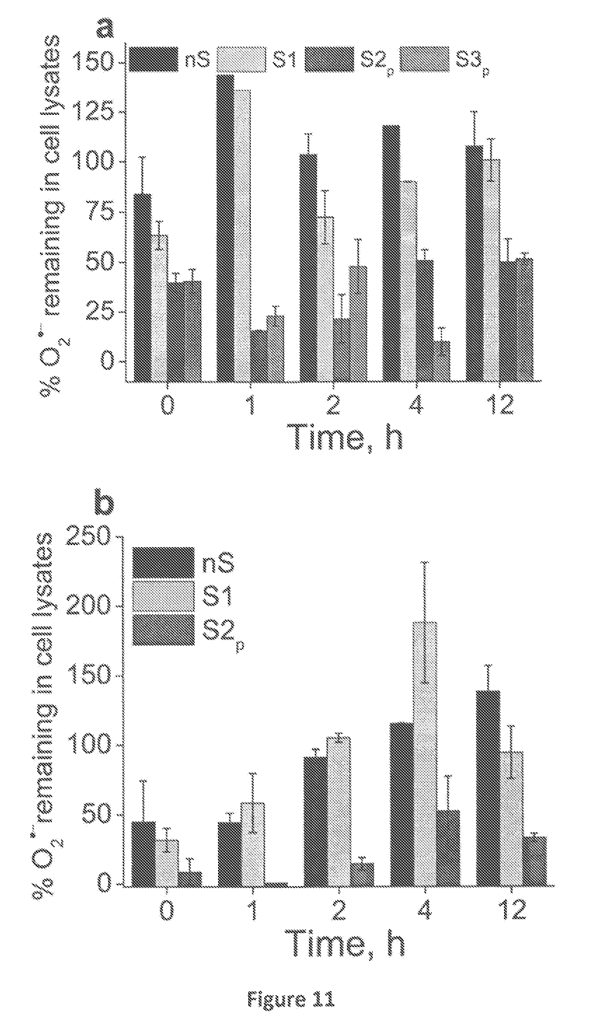

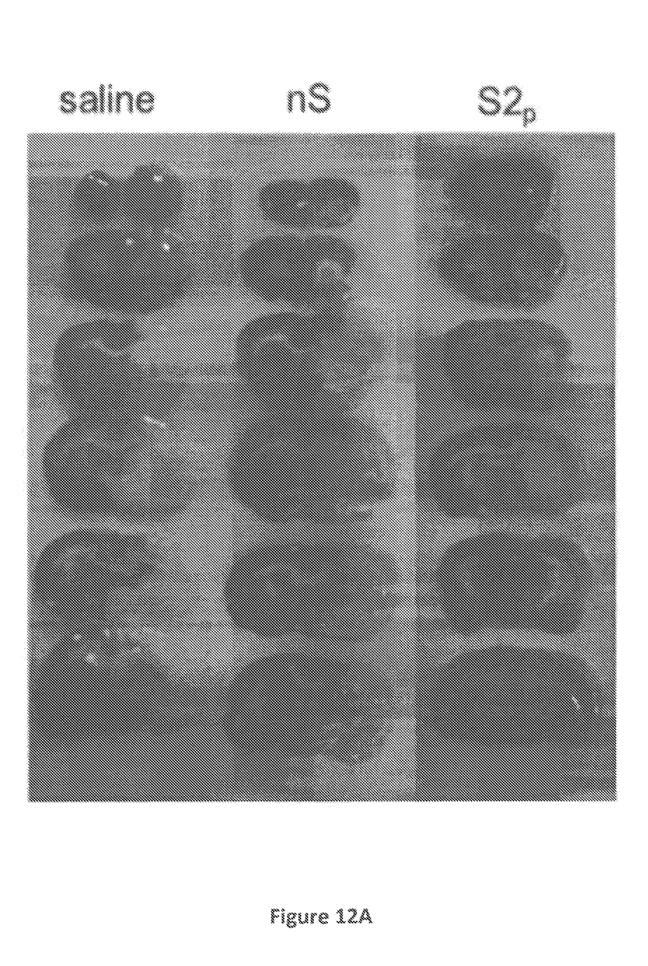

The ability of SOD 1 formulations to scavenge experimentally induced O₂.⁻ was determined in cells pre-treated with different formulations (FIG. 11). Both TBMEC and CATH.a neurons treated with purified cl-nanozymes (S2p and S3p) displayed greater ability to scavenge O₂ compared to cells treated with native SOD1 and non-cross-linked BIC (nS and S1). This effect lasted for at least 12 hours post-treatments with S2p and S3p.

### Therapeutic Efficacy In Vivo in a Rat MCAO Model of Stroke (Ischemia/Reperfusion Injury)

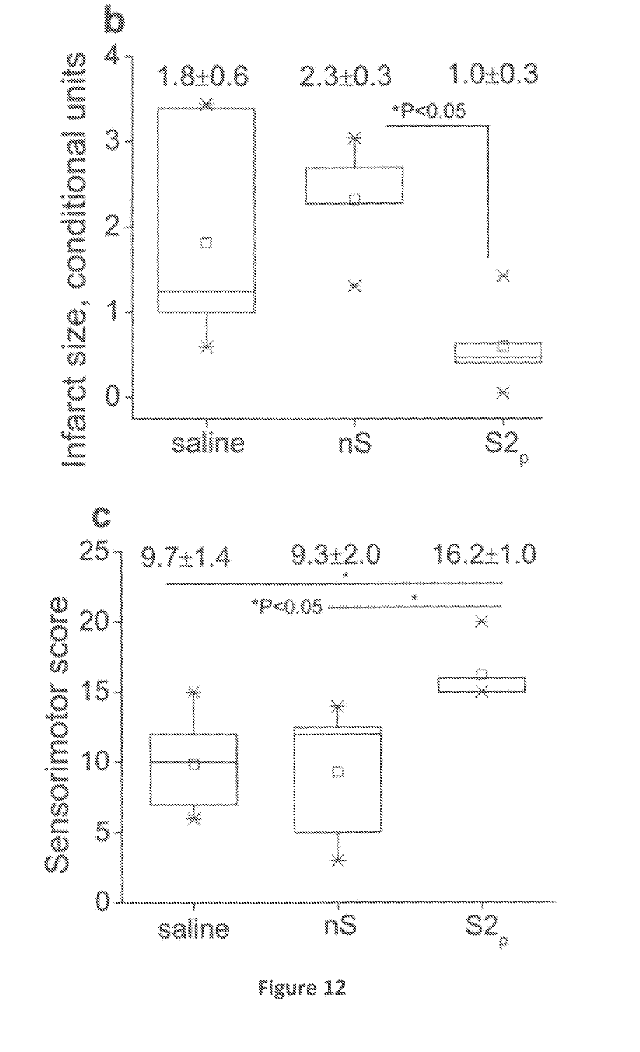

The proof of therapeutic efficacy was shown in a rat MCAO model of stroke. In this model ischemia/reperfusion injury is associated with overproduction of ROS that predominantly cause tissue damage (Reddy et al. (2009) FASEB J., 23:1384-1395). Hence, ROS scavenging by purified SOD1 cl-nanozymes can result in attenuation of oxidative damage and produce a therapeutic response. To assess the extent of brain injury after different treatments, TTC staining was used as a simple and quick method for determining the infarct size (Benedek et al. (2006) Brain Res., 1116:159-165). In the viable brain tissue TTC is enzymatically reduced by dehydrogenases to a red formazan product while pale staining corresponds to infarct areas (FIG. 12A). Rats treated with purified cl-nanozymes (S2p) showed decreased apparent infarct size in the ipsilateral hemisphere, compared to those treated with saline/native SOD1 (nS). Image analysis and quantification of the brain slices indicated a 59% reduction in infarct volume (FIG. 12B). Furthermore, the analysis of the sensorimotor functions of rats revealed a significant 70% improvement in the functional outcomes (FIG. 12C) after a single i.v. injection of purified cl-nanozymes at a dose of 10 kU/kg compared to native SOD1.

Hematoxylin and eosin (H&E) staining of peripheral organs also showed that no toxicity associate with nanozyme treatment was observed in the rat MCAO model of stroke at 24 hours. The biodistribution of native SOD1, non-purified cl-nanozymes, and purified cl-nanozymes were observed, using ¹²⁵I labeling by IODO-BEADS, upon administration to healthy mice. Notably, the three agents demonstrated unique biosdistributions. While all three showed similar levels in blood, native SOD1 was predominantly located in the kidney while purified cl-nanozymes was localized more heavily in the spleen and liver. Non-purified cl-nanozymes had a biodistribution between native SOD1 and purified cl-nanozymes. All three agents also showed some presence in the lungs, with purified cl-nanozymes exhibiting the greatest amount. 3,3′-Diaminobenzidine (DAB) and fluorescent staining (using anti-PEG primary antibodies) confirmed these results. Notably, double staining for ED1 (CD68) and PEG revealed cl-nanozymes in kidney and liver associated with phagocytes. In liver, cl-nanozymes exhibit association with hepatocytes in addition to Kupffer cells, although much cl-nanozymes is not associated with cells, with some in sinusoids and some in bile canaliculi. In the brain of animals with stroke, cl-nanozymes were observed in association with phagocytes (although very few phagocytes are present in the brain parenchyma at 3 hours post reperfusion onset) and in association with vasculature—and only in the area of infarct.

cl-nanozymes have been reported with a cross-linked polyelectrolyte complex core stabilized by amide bonds between the carboxylic groups of SOD 1 and the amino groups of PEG-pLL₅₀. Such cl-nanozymes display improved delivery to the brain and are more stable in blood and brain tissues compared to the non cross-linked SOD1 BICs (Klyachko et al. (2012) Nanomed. Nanotechnol. Biol. Med., 8:119-129). Herein, the amino groups in the polycation template were cross-linked. To minimize the possibility of the side reactions with protein amino groups, an excess of the polycation was used (Z=2), which based on ζ-potential measurements results in formation of BICs containing some excess of pLL amines. These amines can react with the cross-linking agents, e.g. DTSSP or BS³. This approach results in lowering the extent of modification of enzyme reactive groups compared to the core cross-linking strategy. Indeed SOD1 cl-nanozymes retained ≧90% activity indicating that protein lysines were mostly spared during the crosslinking reaction as their modification is known to inactivate the enzyme (Cocco et al. (1982) FEBS Lett., 150:303-306). Moreover, using a different cross-linker, dimethyl 3,3′-dithiobispropionimidate (DTBP) led to a loss of 30 to 80% of SOD1 activity (Table 5), probably due to extensive modification of protein lysines.

TABLE 5
Characteristics of DTBP cross-linked SOD1 nanozymes.

| | | | Enzyme Activity, |
|---|---|---|---|
| Sample^{a,b} | D_{eff}, nm | PDI^{c} | % of Initial |
| nS | 5.3 | 0.2 | 100 |
| S1^{d} | 8.1 | 0.1 | 93 |
| S4(0.5) | 14.5 | 0.2 | 67 |
| S4(1.0) | 11.8 | 0.2 | 31 |
| S4(2.5) | 8.9 | 0.1 | 18 |

^{a}nS—native SOD1, S1—non-cross-linked BICs of SOD1, S4—cross-linked cl-nanozymes; numbers in parentheses indicate molar ratio of DTBP/pLL amines,
^{b}All samples (1 mg/mL) were 10 mM HEPES, pH 7.4,
^{c}polydispersity index;
^{d}Z = 2.

Gel retardation analysis indicated that DTSSP was more efficient in cross-linking than BS³. DTSSP is more hydrophilic than BS³—their octanol-water partition coefficients (log P) are −2.1 and −1, respectively. This alone may result in better reactivity of DTSSP towards the hydrophilic amino groups. There was an additional indication that different cross-linkers result in different cl-nanozyme formats: while the particle size increased after cross-linking with DTSSP or BS³ (Table 1) it did not change after cross-linking with DTBP (Table 5). Interestingly, a published report (Oupicky et al. (2001) Gene Ther., 8:713-724) indicated that DTSSP-crosslinked pLL/pDNA polyplexes also demonstrated increased particle size, while no such increase was observed in case of DTBP. The sizes may increase due to cross-linking of multiple BIC particles although this does not seem to be reflected in AFM images that display separated spheres for both non-cross-linked and cross-linked BICs. The BICs are dynamic formations, which constantly exchange their polyionic components (Li et al. (2008) Macromolecules, 41:5863-5868). In the presence of the cross-linker such polyion components may become covalently immobilized in the “host” BICs resulting in particle growth. This will depend on the reactivity of the cross-linker—the growth is more likely for less reactive agents, which form longer living “transitory states”, than for highly reactive agents that tend to rapidly fix the existing structures. The higher reactivity of DTBP compared to DTSSP and BS³ could therefore be responsible for lack of particle enlargement as well as loss of enzyme activity.

Physicochemical characteristics such as particle size, surface charge and morphology influence in vivo disposition of nanoparticles (Choi et al. (2009) Nano Lett., 9:2354-2359; Choi et al. (2007) Nat. Biotechnol., 25:1165-1170). Purification of cl-nanozymes resulted in improved homogeneity of the samples as demonstrated by gel retardation, DLS and AFM. Purified SOD1 cl-nanozyme showed a PDI of <0.05 indicating near monodisperse particles. Purified catalase cl-nanozyme also had particles with unimodal distribution and narrower PDI compared to non-purified cl-nanozyme. The small size (<100 nm), narrower size distribution and chemical homogeneity of the purified cl-nanozymes will decrease their uptake by the reticuloendothelial system (RES), increase their in vivo stability and decrease clearance. Sedimentation equilibrium analysis also demonstrated that purified cl-nanozymes contained no aggregates, which favors avoidance of RES uptake. Native SOD 1 has a short circulation half-life of 6 minutes (Davis et al. (2007) Neurosci. Lett., 411:32-36) and is rapidly cleared from circulation in addition to inactivation by ubiquitous proteases. SOD1 cl-nanozyme will circulate longer and be protected against proteolytic degradation.

The cell line models in the instant studies represent key cell types of the neurovascular unit. They are likely targets in treating cerebrovascular diseases including stroke given that both neurons and microvessels respond equally rapidly to the ischemic insult (del Zoppo, G. J. (2006) N. Engl. J. Med., 354:553-555; Mabuchi et al. (2005) J. Cereb. Blood Flow Metab., 25:257-266). Decreased cytotoxicity of purified SOD1 cl-nanozymes in these cells will allow administration of their higher doses. The choice of the cross-linking agent may also be considered since the BS³-cross-linked cl-nanozymes are less toxic than DTSSP-cross-linked cl-nanozymes. Subcellular reduction of disulfide bonds in DTSSP links may lead to release of the polycationic species that display toxicity. However, it should be noted that upon complete degradation of the block copolymer, lysines will be metabolized to acetyl-coenzyme A (acetyl-CoA) or acetoacetyl-CoA in vivo and PEG is biocompatible.

The prolonged antioxidant effect of purified SOD1 cl-nanozyme in cells (up to 12 hours post-withdrawal of the treatment) is most likely due to its improved stability. The PEG-pLL₅₀ chains in the BIC can sterically protect SOD1 molecules against degradation by intracellular proteases. This is further supported by the fact that in spite of the lower uptake of cl-nanozymes compared to non-cross-linked BIC, the internalized fraction remained more catalytically active over time. This also relates well to the observation that cl-nanozyme not only delivered higher amounts of SOD1 to the brain parenchyma, but was also retained to a higher extent in brain capillaries in healthy mice (Klyachko et al. (2012) Nanomed. Nanotechnol. Biol. Med., 8:119-129). Higher retention in the brain capillaries allows the use of such nanoparticles to treat cerebrovascular disorders like stroke where rescue of the BBB from oxidative damage results in therapeutic outcomes (del Zoppo, G. J. (2006) N. Engl. J. Med., 354:553-555). This led to the testing of the potential of purified SOD1 cl-nanozymes to treat stroke in a rat MCAO model.

Using this model, a clear decrease in the infarct volume was observed concomitant with significant improvement in the sensorimotor function after i.v. injection of a single dose of purified SOD1 cl-nanozyme. Its therapeutic efficacy may be due to ROS scavenging both at the level of the brain microvessels and brain parenchyma. The former could be explained by the increased retention and stability of the SOD1 cl-nanozyme in the BBB. In addition, the compromised integrity of the BBB, a well-known phenomenon in CNS pathologies (including stroke (Nagaraja et al. (2008) Microcirculation 15:1-14)), may also improve delivery of the cl-nanozymes to neurons and supportive cells (astrocytes, glial cells and resident inflammatory cells) resulting in their protection from oxidative stress. Thus, both improved accumulation of the SOD1 cl-nanozymes due to BBB permeability and increased retention of active enzyme in the brain microvessels could contribute to decreased brain injury upon stroke.

Notwithstanding therapeutic effect of cationic liposomes (Imaizumi et al. (1990) Stroke 21:1312-1317; Chan et al. (1987) Ann. Neurol., 21:540-547), their translational significance might be limited due to low stability as pharmaceutical formulations (Sinha et al. (2001) Biomed. Pharmacother., 55:264-271). Contrary to SOD1 liposomes, covalently stabilized cl-nanozymes are stable, and in this regard represent innovative formulations. Toxicity is another concern for cationic carriers (including cationic liposomes) and this is addressed herein by developing nearly electroneutral forms of cl-nanozymes with considerably decreased toxicity to brain endothelial cells and neurons. While cellular entry of PEG-SOD1 is a limitation (Veronese et al. (2002) Adv. Drug Deliv. Rev., 54:587-606), cl-nanozyme enters cells of the neurovascular unit, which is beneficial for treatment.

In contrast to PLGA nanoparticles that gradually release encapsulated SOD1 over days and weeks, SOD1 in cl-nanozyme formulations is fully and immediately available for O₂.⁻ scavenging. While PLGA hydrolysis may affect stability of encapsulated proteins (Jiang et al. (2008) Mol. Pharm., 5:808-817), SOD1 remains stable in cl-nanozyme formulation as indicated by the sustained decomposition of O₂.⁻ in the in vitro studies. In contrast to PLGA particles that were injected i.c., the cl-nanozymes demonstrated therapeutic efficacy after i.v. injection. Indeed, administration of purified cl-nanozyme suppressed brain tissue damage and also improved sensorimotor functions.

Herein, a simple method to prepare well-defined cross-linked antioxidant nanozymes containing SOD 1 or catalase was developed and their physicochemical properties were characterized. The ability of such constructs to scavenge O2.⁻ radicals in vitro in two cell culture models—cultured brain microvessel endothelial cells and central neurons—was validated. Further, it was demonstrated that SOD1 cl-nanozymes can attenuate oxidative damage, induce tissue protection and improve functional outcomes in a rat MCAO model of ischemia/reperfusion injury.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of synthesizing a nanozyme comprising a therapeutic protein, said method comprising
a) complexing at least one block copolymer and at least one therapeutic protein, wherein said block copolymer comprises at least one ionically charged polymeric segment and at least one hydrophilic polymeric segment;
b) cross-linking said block copolymer with said therapeutic protein by contacting the complex of step a) with a cross-linker, thereby generating nanozymes; and
c) purifying the nanozymes of step b).

2. The method of claim 1, wherein said therapeutic protein is an antioxidant enzyme.

3. The method of claim 1, wherein said cross-linker forms an amide bond between an amino group of the ionically charged polymeric segment and a carboxylic group of the therapeutic protein.

4. The method of claim 1, wherein said cross-linker forms a bond between amino groups of the polymeric segment or between an amino group of the ionically charged polymeric segment and an amino group of the therapeutic protein.

5. The method of claim 1, wherein said cross-linker is 3,3′-dithiobis(sulfosuccinimidylpropionate) (DTSSP) or bis(sulfosuccinimidyl)suberate (BS³).

6. The method of claim 1, wherein the molar ratio of cross-linker to said ionically charged polymeric segment is equal to or less than about 0.5.

7. The method of claim 1, wherein said ionically charged polymeric segment is cationic.

8. The method of claim 6, wherein said cationic polymeric segment comprises poly-lysine.

9. The method of claim 1, wherein said hydrophilic polymeric segment comprises poly(ethylene glycol).

10. The method of claim 2, wherein said antioxidant enzyme is superoxide dismutase or catalase.

11. The method of claim 1, wherein step c) comprises size exclusion chromatography and/or centrifugal filtration.

12. The method of claim 1, wherein the purification in step c) results in nanozymes that are at least about 95% pure.

13. The nanozyme synthesized by the method of claim 1.

14. A composition comprising at least one nanozyme of claim 13 and at least one pharmaceutically acceptable carrier.

15. The composition of claim 14 further comprising at least one other antioxidant.

16. A method of treating a reactive oxygen species (ROS)-related disease or disorder in a subject in need thereof, said method comprising administering at least one composition of claim 14 to the subject.

17. The method of claim 16, wherein said reactive oxygen species (ROS)-related disease or disorder is selected from the group consisting of stroke, hypertension, heart failure, arthritis, cancer, cardiovascular diseases, atherosclerosis, autoimmune disease, ischemia/reperfusion injury, traumatic brain injury, restenosis, inflammation, lung inflammation associated with influenza infection, acute respiratory distress syndrome (ARDS), asthma, inflammatory bowel disease (IBD), a dermal and/or ocular inflammation, metabolic disease or disorder, obesity, diabetes, neurological disorders, multiple sclerosis, cerebral palsy, HIV-associated dementia, neurocardiovascular disease/dysregualtion, neurodegenerative disease or disorder, Alzheimer's disease, Huntington's disease, Parkinson's disease, Lewy Body disease, amyotrophic lateral sclerosis, and prion disease.

18. The method of claim 16, wherein said ROS-related disease or disorder is stroke.
